SEQUENCE LISTING (12 sequences)

SEQ ID NO: 1 - protein, 9 aa, Artificial Sequence
  Synthetic construct
THQGLSSPV

SEQ ID NO: 2 - protein, 9 aa, Artificial Sequence
  Synthetic construct
TKGPSVFPL

SEQ ID NO: 3 - protein, 7 aa, Artificial Sequence
  Synthetic construct
QSHSWPF

SEQ ID NO: 4 - protein, 7 aa, Artificial Sequence
  Synthetic construct
IRSKSIN

SEQ ID NO: 5 - protein, 9 aa, Artificial Sequence
  Synthetic construct
YSLSSTLTL

SEQ ID NO: 6 - protein, 9 aa, Artificial Sequence
  Synthetic construct
TQSPAILSV

SEQ ID NO: 7 - protein, 9 aa, Artificial Sequence
  Synthetic construct
STYSLSSTL

SEQ ID NO: 8 - protein, 9 aa, Artificial Sequence
  Synthetic construct
VQWKVDNAL

SEQ ID NO: 9 - protein, 9 aa, Artificial Sequence
  Synthetic construct
CQQSHSWPF

SEQ ID NO: 10 - protein, 9 aa, Artificial Sequence
  Synthetic construct
LQSSGLYSL

SEQ ID NO: 11 - protein, 9 aa, Artificial Sequence
  Synthetic construct
STSGGTAAL

SEQ ID NO: 12 - protein, 9 aa, Artificial Sequence
  Synthetic construct
ACEVTHQGL

(19) United States
(12) Patent Application Publication
(10) Pub. No.: US 2022/0062372 A1
(43) Pub. Date: Mar. 3, 2022

(54) METHODS FOR TREATING DISEASE AND REDUCING DRUG-INDUCED LIVER INJURY IN PATIENT POPULATIONS

(71) Applicant: Ten Peaks LLC, Corona Del Mar, CA (US)

(72) Inventors: Sundar SRINIVASAN, Corona Del Mar, CA (US); Christina Chow WALLEN, Seattle, WA (US)

(21) Appl. No.: 17/464,175

(22) Filed: Sep. 1, 2021

Related U.S. Application Data
(60) Provisional application No. 63/073,044, filed on Sep. 1, 2020.

(51) Int. Cl. A61K 38/08; A61K 39/395; A61K 38/21; A61K 38/17; A61K 31/137; A61K 31/136; A61K 31/52; A61K 31/277; A61K 31/225; A61K 31/519; A61K 38/13; A61P 1/16

(52) U.S. Cl. CPC A61K 38/08; A61P 1/16; A61K 38/212; A61K 38/215; A61K 38/177; A61K 31/137; A61K 31/136; A61K 31/52; A61K 31/277; A61K 31/225; A61K 31/519; A61K 38/1774; A61K 38/1793; A61K 38/13; A61K 39/3955

(57) ABSTRACT

The present disclosure provides methods of reducing the risk of developing, and/or severity of, an adverse drug reaction such as drug-induced liver injury (DILI). The methods include identifying patients at risk for developing DILI by determining the presence or absence of one or more HLA alleles in the patients.

### CROSS-REFERENCE TO RELATED APPLICTIONS

This application claims the benefit of priority to U.S. Provisional Application No. 63/073,044, filed on Sep. 1, 2020, the entire contents of each of which are incorporated by reference.

### DESCRIPTION OF THE TEXT FILE SUBMITTED ELECTRONICALLY

The contents of the text file submitted electronically herewith are incorporated herein by reference in their entirety: A computer readable format copy of the Sequence Listing (filename: MSLC-009_01US_SeqList_ST25, recorded Sep. 1, 2021, file size 2573 bytes.

### BACKGROUND

Adverse drug reactions (ADRs) can occur in patients who are treated with therapeutic agents. In particular, drug-induced liver injury (DILI) is a serious ADR that can lead to acute liver failure. Upon diagnosis of DILI, administration of the therapeutic drug that triggered the DILI is generally either reduced or discontinued, but often DILI is not detected early enough to avoid long-term or permanent liver damage, or even death.

There is no information currently available to help assess a patient population's risk of developing DILI upon treatment with therapeutic drugs. There is a need in the art to predict, manage, and prevent the development of ADRs such as DILI in patients that have been or may otherwise be prescribed a drug that causes the dangerous reaction in a certain patient population. This disclosure addresses this and other needs.

### SUMMARY OF THE INVENTION

The present disclosure provides for methods of treating a subject or patient population suffering from an autoimmune, inflammatory, or other disease that may be amenable to treatment with a therapeutic drug, while reducing the risk and/or severity of drug-induced liver injury (DILI). In embodiments, the disclosure provides methods for decreasing the risk of, decreasing the incidence of, decreasing the severity of, and/or ameliorating the symptoms of DILI. The present inventors found that the presence of a particular HLA allele in a subject confers to the subject a higher risk of the development of DILI upon administration of a drug having one or more particular peptides. Accordingly, the methods provided herein involve identifying the at-risk subject or patient population, and reducing the risk of development and/or reducing the severity of DILI in the subject or patient population. In addition, the methods provided herein involve a safe method for treating a subject with a drug having one or more of the peptides provided herein, by determining the presence or absence of a particular HLA allele prior to treatment. In embodiments, the HLA allele is HLA-B*39:01.

In one aspect, the present disclosure provides methods for treating a disease or disorder in a subject in need thereof, comprising administering a therapeutic drug to the subject in an amount effective to treat the disease or disorder. In another aspect, the present disclosure provides methods for reducing the risk of drug-induced liver injury (DILI) in a subject while treating the subject for disease or disorder with a therapeutic drug effective to treat the disease or disorder. In embodiments, the disease or disorder is an autoimmune disease and/or an inflammatory disease and/or a cancer. For example, in embodiments, the cancer is a leukemia or lymphoma. In embodiments, the subject has an HLA profile comprising HLA-B*39:01. In embodiments, the therapeutic drug is a protein comprising an amino acid sequence selected from the group consisting of SEQ ID NOs: 1-11. In embodiments, the methods comprise administering to the subject a therapeutic agent for the treatment of the disease or disorder, wherein the therapeutic agent does not comprise one or more amino acid sequence selected from the group consisting of SEQ ID NO: 1-11.

In one aspect, the present disclosure provides methods for treating a disease or condition in a subject in need of a therapy; and/or methods for reducing the risk of DILI in a subject, comprising first determining that the subject has an HLA profile that does not include an HLA-B*39:01 allele, then administering to the subject a therapeutically effective amount of a therapeutic agent, wherein the therapeutic agent is a protein that comprises an amino acid sequence according to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or any combination thereof. In another aspect, the present disclosure provides methods for treating a disease or condition in a subject in need of a therapy; and/or methods for reducing the risk of DILI in a subject, comprising: (i) identifying the subject as not having a genetic variation comprising the HLA-B*39:01 allele; and (ii) administering a therapeutically effective amount of a therapeutic agent to the subject, wherein the therapeutic agent is a protein that comprises an amino acid sequence according to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or any combination thereof. In further embodiments, the subject has a decreased risk of drug-induced liver injury (DILI) compared to an otherwise identical subject having the genetic variation. In yet another aspect, the present disclosure provides methods for treating a subject having a disease or condition with a therapeutic agent, wherein the therapeutic agent is a protein that comprises an amino acid sequence according to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or any combination thereof, the method comprising the steps of: (i) obtaining or having obtained the HLA profile of the subject; and (ii) if the HLA profile of the subject does not comprise HLA-B*39:01, then administering the therapeutic agent to the subject, and if the HLA profile of the subject comprises HLA-B*39:01, then administering to the subject an alternative therapeutic that does not comprise an amino acid sequence according to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, or SEQ ID NO: 11.

In embodiments, the therapeutic agent comprising an amino acid sequence according to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or any combination thereof is an antibody or antibody fragment. In embodiments, the therapeutic agent comprising an amino acid sequence according to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or any combination thereof is selected from the group consisting of abciximab, adalimumab, alemtuzumab, alirocumab, atezolizumab, atoltivimab, maftivimab, odesivimab, basiliximab, belantamab mafodotin, belimumab, benralizumab, bevacizumab, bezlotoxumab, brentuximab vedotin, brodalumab, brolucizumab, burosumab, canakinumab, capromab pendetide, cemiplimab, certolizumab pegol, cetuximab, crizanlizumab, daclizumab, daratumumab, denosumab, dinutuximab, dupilumab, durvalumab, eculizumab, efalizumab, elotuzumab, emapalumab, emicizumab, enfortumab vedotin, erenumab, evolocumab, fanolesomab, fremanezumab, galcanezumab, gemtuzumab ozogamicin, guselkumab, ibalizumab, ibritumomab tiuxetan, idarucizumab, inebilizumab, infliximab, inotuzumab ozogamicin, ipilimumab, isatuximab, ixekizumab, lanadelumab, mepolizumab, mogamulizumab, natalizumab, naxitamab, necitumumab, nivolumab, nofetumomab, obiltoxaximab, obinutuzumab, ocrelizumab, ofatumumab, olaratumab, omalizumab, panitumumab, pembrolizumab, pertuzumab, polatuzumab vedotin, ramucirumab, ranibizumab, ravulizumab, raxibacumab, reslizumab, risankizumab, rituximab, romosozumab, sacituzumab govitecan, sarilumab, satralizumab, secukinumab, siltuximab, tafasitamab, teprotumumab, tocilizumab, tositumomab, trastuzumab, ustekinumab, vedolizumab, tisotumab vedotin, epcoritamab, amivantamab, teclistamab, camidanlumab tesirine, talquetamab, actimab-a, lintuzumab. In embodiments, the therapeutic agent comprising an amino acid sequence according to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or any combination thereof comprises two, three, four, five, six, seven, eight, nine, or ten sequences selected from SEQ ID NOs. 1-11. In embodiments, the therapeutic agent comprising an amino acid sequence according to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or any combination thereof comprises all eleven of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, and SEQ ID NO: 11.

In one aspect, the present disclosure provides methods for treating a disease or condition in a subject in need thereof, wherein the subject has an HLA profile comprising HLA-B*39:01, wherein the method comprises (i) determining that the subject has an HLA profile comprising HLA-B*39:01 by obtaining or having obtained the HLA profile of the subject, and (ii) administering to the subject a therapeutically effective amount of a therapeutic agent for the treatment of the autoimmune disease, wherein the therapeutic agent is not a protein that comprises an amino acid sequence according to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, or SEQ ID NO: 11. In another aspect, the present disclosure provides methods for reducing the risk of DILI in a subject, wherein the subject has an HLA profile comprising HLA-B*39:01, wherein the method comprises (i) determining that the subject has an HLA profile comprising HLA-B*39:01 by obtaining or having obtained the HLA profile of the subject, and (ii) administering to the subject a therapeutically effective amount of a therapeutic agent for the treatment of the autoimmune disease, wherein the therapeutic agent is not a protein that comprises an amino acid sequence according to any of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, or SEQ ID NO: 11. In another aspect, the present disclosure provides methods for treating a population of patients having a disease or condition, the method comprising (i) providing a first population of patients having the disease or disorder; (ii) excluding only patients having an HLA profile comprising HLA-B*39:01 from the first population of patients; (iii) treating the population of patients not excluded from the first population with a therapeutically effective amount of a therapeutic agent, wherein the therapeutic agent is a protein comprising an amino acid sequence according to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or any combination thereof; and (iv) treating the excluded patients with a therapeutically effective amount of an alternative therapeutic agent, wherein the alternative therapeutic agent is not a protein comprising an amino acid sequence according to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, or SEQ ID NO: 11. In another aspect, the present disclosure provides methods for reducing the risk of DILI and/or the incidence of DILI in a population of patients, the method comprising (i) providing a first population of patients having a disease or disorder; (ii) excluding only patients having an HLA profile comprising HLA-B*39:01 from the first population of patients; (iii) treating the population of patients not excluded from the first population with a therapeutically effective amount of a therapeutic agent, wherein the therapeutic agent is a protein comprising an amino acid sequence according to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or any combination thereof; and (iv) treating the excluded patients with a therapeutically effective amount of an alternative therapeutic agent, wherein the alternative therapeutic agent is not a protein comprising an amino acid sequence according to any of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, or SEQ ID NO: 11.

In another aspect, the present disclosure provides methods for treating a disease or condition in a subject in need of a therapy, comprising: (i) identifying the subject as having a genetic variation comprising the HLA-B*39:01 allele; and (ii) administering a therapeutically effective amount of a therapeutic agent to the subject, wherein the therapeutic agent is not a protein that comprises an amino acid sequence according to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, or SEQ ID NO: 11. In another aspect, the present disclosure provides methods for treating a disease or condition in a subject in need of a therapy, comprising first determining that the subject has an HLA profile that includes an HLA-B*39:01 allele, then administering to the subject a therapeutically effective amount of a therapeutic agent, wherein the therapeutic agent is not a protein that comprises an amino acid sequence according to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, or SEQ ID NO: 11.

In another aspect, the present disclosure provides methods for reducing the risk of DILI in a subject, comprising: (i) identifying the subject as having a genetic variation comprising the HLA-B*39:01 allele; and (ii) administering a therapeutically effective amount of a therapeutic agent to the subject, wherein the therapeutic agent is not a protein that comprises an amino acid sequence according to any of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, or SEQ ID NO: 11. In another aspect, the present disclosure provides methods for reducing the risk of DILI in a subject, comprising first determining that the subject has an HLA profile that includes an HLA-B*39:01 allele, then administering to the subject a therapeutically effective amount of a therapeutic agent, wherein the therapeutic agent is not a protein that comprises an amino acid sequence according to any of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, or SEQ ID NO: 11.

In embodiments, the therapeutic agent that does not comprise an amino acid sequence according to any of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, or SEQ ID NO: 11 is selected from the group consisting of azathioprine, mercaptopurine, methotrexate, mesalamine, budesonide, hyoscyamine, celecoxib, hydroxychloroquin, etanercept, prednisone, cyclosporine, meloxicam, leflunomide, sulfasalazine, abatacept, avelumab, blinatumomab, caplacizumab, golimumab, moxetumomab pasudotox, palivizumab, tildrakizumab, acitretin, apremilast, corticotropin, interferon-alpha, interferon-beta, glatiramier acetate, fingolimod, mitoxanthrone, azathioprine, teriflunomide, and dimethylfumarate.

In embodiments, the autoimmune disease is multiple sclerosis, and the therapeutic agent that does not comprise an amino acid sequence according to any of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, or SEQ ID NO: 11 is selected from the group consisting of interferon-alpha, interferon-beta, glatiramier acetate, fingolimod, mitoxanthrone, azathioprine, teriflunomide, dimethylfumarate.

In embodiments, the autoimmune disease is rheumatoid arthritis, ankylosing spondylitis, or psoriatic arthritis, and the therapeutic agent that does not comprise an amino acid sequence according to any of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, or SEQ ID NO: 11 is selected from the group consisting of methotrexate, abatacept, etanercept, and golimumab.

In embodiments, the autoimmune disease is Crohn's disease or ulcerative colitis, and the therapeutic agent that does not comprise an amino acid sequence according to any of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, or SEQ ID NO: 11 is selected from the group consisting of methotrexate, azathioprine, cyclosporine, and mercaptopurin.

In embodiments provided herein, the disease or condition is an autoimmune disease, an inflammatory disease, or a cancer. In embodiments, the autoimmune disease or inflammatory disease is selected from the group consisting of Crohn's Disease, Ulcerative Colitis; rheumatoid arthritis, polyarticular or systemic juvenile idiopathic arthritis, ankylosing spondylitis; psoriatic arthritis, plaque psoriasis, psoriasis vulgaris, pustular psoriasis, erythrodermic psoriasis uveoretinitis, panuveitis, Behcet's disease; pustular psoriasis, erythrodermic psoriasis, hidradenitis suppartiva; panuveitis, granulomatosis with polyangitis (Wegner's granulomatosis), systemic lupus erythematosus, and multiple sclerosis.

In embodiments, the disease or condition is a cancer, such as a lymphoma. In embodiments, the cancer is a B cell lymphoma or a T cell lymphoma. In embodiments, the cancer is selected from non-Hodgkin's Lymphoma (NHL), chronic lymphocytic leukemia (CLL), follicular lymphoma, and diffuse large B-cell lymphoma.

In embodiments, the methods provided herein comprise obtaining or having obtained the HLA profile of a subject or of a population of subjects. In embodiments, the methods comprise obtaining or having obtained a biological sample from the subject in order to test the biological sample and obtain information regarding the subject's HLA profile. In embodiments, the methods comprise performing a genetic assay to determine the presence or absence of the HLA-B*39:01 allele. In embodiments, the genetic assay comprises one or more of a polymerase chain reaction (PCR)-based approach, a direct sequencing approach, a next generation (NGS) approach and/or a direct HLA typing test. In embodiments, the genetic assay comprises obtaining a PCR-amplified genomic DNA sample of the biological sample from the subject, contacting under hybridizing conditions the genomic DNA with an oligonucleotide that specifically hybridizes to HLA-B*39:01, and detecting the presence of HLA-B*39:01 in the sample.

### BRIEF DESCRIPTION OF THE FIGURES

FIG. 1 shows a summary of the alleles with significant results in the cases vs. controls individual data sets.

FIG. 2A-2D shows the binding of a standard (i.e., protein of known molecular weight; FIG. 2A), SEQ ID NO: 1 (FIG. 2B), SEQ ID NO: 2 (FIG. 2C), or a control peptide, SEQ ID NO: 12 (FIG. 2D) to HLA-B*039:01.

FIG. 3 shows the binding of SEQ ID NO: 1 (3901_thq), SEQ ID NO: 2 (3901 tkg), control peptide SEQ ID NO: 12 (3901_ace) or a standard to HLA-B*039:01 in a single graph.

FIG. 4A-4C each show the binding of control peptide SEQ ID NO: 12 (ace), SEQ ID NO: 1 (thq) and SEQ ID NO: 2 (tkg) to HLA-B*057:01 (FIG. 4A), HLA-B*15:01 (FIG. 4B), and HLA-B*39:01; same data as shown in FIGS. 2 and 3). FIG. 4D shows the binding of a standard, no peptide, or control peptide.

FIG. 5A shows the crystal structure of infliximab. SEQ ID NO: 1 is circled. FIG. 5B shows a closer view of SEQ ID NO: 1 (circled).

### DETAILED DESCRIPTION

The present disclosure is related to the inventors' discovery that DILI in subjects treated with certain drugs is associated with particular genetic variants; and that the particular genetic variants interact with one or more specific amino acid sequences present in the drug or drugs. The present inventors discovered that a defined HLA profile is associated with DILI following treatment with certain drugs. In embodiments, the HLA-B*39:01 allele was associated with a high frequency of DILI after administration of infliximab. Further, without wishing to be bound by theory, the present inventors discovered that certain amino acids present in infliximab cause the interaction between infliximab and the HLA-B*39:01 allele. In embodiments, other therapeutic agents comprising certain amino acid sequences similarly interact with the HLA-B*39:01 allele to cause DILI. In embodiments, the therapeutic agents comprising one or more of the certain amino acid sequences are therapeutic agents used for the treatment of one or more autoimmune disease, inflammatory disease, and/or cancer. Accordingly, the present disclosure provides improved methods for treatment of diseases (e.g., autoimmune diseases, inflammatory diseases, and cancers) in a defined patient population, and/or for reduction in the risk of DILI in the defined patient population.

### Definitions

As used herein, the term “about” refers to an amount somewhat more or less than the stated parameter value, for example plus or minus five or ten percent of the object that “about” modifies, or as one of skill in the art would recognize from the context (e.g., approximately 50% of the interval between values). The term “about” also includes the value referenced. For example, a dose of about 1 mg includes 1 mg, as well as values somewhat below or above 1, such as 0.9 mg and 1.1 mg. As used herein, “and/or” refers to and encompasses any and all possible combinations of one or more of the associated listed items, as well as the lack of combinations when interpreted in the alternative (“or”).

As used herein, the term “patient” refers to a human. In embodiments, the patient can be a male or a female. In embodiments, the patient can be an adult, or a pediatric patient. The term “subject” is used interchangeably herein with “patient.” In embodiments, the subject is a human that has a disease or condition that may be treated with a therapeutic agent provided herein, e.g., a therapeutic agent comprising one or more of the amino acid sequences set forth in SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or any combination thereof. For example, the subject is a human that has a disease or condition wherein a therapeutic agent having one or more of SEQ ID NOs: 1-11 is approved by a regulatory agency for use in the disease or condition, and/or is used off-label to treat the disease or condition. The therapeutic agent having one or more of SEQ ID NOs: 1-11 may be selected, for example, from the group consisting of abciximab, adalimumab, alemtuzumab, alirocumab, atezolizumab, atoltivimab, maftivimab, odesivimab, basiliximab, belantamab mafodotin, belimumab, benralizumab, bevacizumab, bezlotoxumab, brentuximab vedotin, brodalumab, brolucizumab, burosumab, canakinumab, capromab pendetide, cemiplimab, certolizumab pegol, cetuximab, crizanlizumab, daclizumab, daratumumab, denosumab, dinutuximab, dupilumab, durvalumab, eculizumab, efalizumab, elotuzumab, emapalumab, emicizumab, enfortumab vedotin, erenumab, evolocumab, fanolesomab, fremanezumab, galcanezumab, gemtuzumab ozogamicin, guselkumab, ibalizumab, ibritumomab tiuxetan, idarucizumab, inebilizumab, infliximab, inotuzumab ozogamicin, ipilimumab, isatuximab, ixekizumab, lanadelumab, mepolizumab, mogamulizumab, natalizumab, naxitamab, necitumumab, nivolumab, nofetumomab, obiltoxaximab, obinutuzumab, ocrelizumab, ofatumumab, olaratumab, omalizumab, panitumumab, pembrolizumab, pertuzumab, polatuzumab vedotin, ramucirumab, ranibizumab, ravulizumab, raxibacumab, reslizumab, risankizumab, rituximab, romosozumab, sacituzumab govitecan, sarilumab, satralizumab, secukinumab, siltuximab, tafasitamab, teprotumumab, tocilizumab, tositumomab, trastuzumab, ustekinumab, vedolizumab, tisotumab vedotin, epcoritamab, amivantamab, teclistamab, camidanlumab tesirine, talquetamab, actimab-a, lintuzumab. The therapeutic agent having one or more of SEQ ID NOs: 1-11 may be selected, for example, from the group consisting of abciximab, adalimumab, alemtuzumab, atezolizumab, basiliximab, belimumab, benralizumab, bevacizumab, bezlotoxumab, burosumab, canakinumab, certolizumab pegol, cetuximab, daclizumab, daratumumab, denosumab, dupilumab, durvalumab, eculizumab, efalizumab, elotuzumab, emicizumab, erenumab, fremanezumab, galcanezumab, gemtuzumab ozogamicin, guselkumab, ibalizumab, ibritumomab tiuxetan, idarucizumab, infliximab, ipilimumab, ixekizumab, lanadelumab, natalizumab, necitumumab, nivolumab, obinutuzumab, ofatumumab, olaratumab, omalizumab, panitumumab, pembrolizumab, pertuzumab, ramucirumab, ranibizumab, ravulizumab, risankizumab, rituximab, sarilumab, secukinumab, siltuximab, tocilizumab, tositumomab, trastuzumab, ustekinumab, and vedolizumab.

In embodiments, prior to being treated with or prescribed a therapeutic agent comprising any one or more of SEQ ID NO:s 1-11 for treatment of the disease or condition, the subject undergoes HLA testing. In other embodiments, a subject that has already been prescribed or begun treatment with a therapeutic agent comprising any one or more of SEQ ID NOs: 1-11 undergoes HLA testing.

As used herein, a “patient population” is a group of patients who may be grouped together by a defining characteristic. For example, in embodiments, the patient population provided herein is a population of patients having an HLA profile provided herein. In further embodiments, the patient population provided herein has an HLA profile provided herein, and is suffering from, at risk for, undergoing treatment for, or being evaluated for treatment for, an autoimmune or inflammatory disease or a cancer. In embodiments, the patient population provided herein has an HLA profile provided herein, and has been treated with, has been prescribed, has been considered for treatment with, may be treated with, or is being evaluated for treatment with, a therapeutic agent that comprises an amino acid sequence according to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or any combination thereof. In embodiments, the patient population provided herein has an HLA profile provided herein, and is suffering from a disease or disorder for which such a therapeutic agent may be administered. In other embodiments, the patient population provided herein is a population from which patients having a particular HLA profile have been excluded. In certain embodiments, the patient population may have other defining characteristics relating to health status, age, sex, race, and/or ethnicity.

As used herein, a “patient treated with” a particular drug or therapy, or a “patient selected for therapy” with a particular drug, or a “patient previously on” the particular drug or therapy and the like refers to a patient having an indication which was amenable to treatment with the particular drug or therapy.

As used herein, “reduce the risk of DILI” and the like means, with respect to a subject, to reduce the likelihood that a subject will develop DILI, and/or to reduce the severity of DILI in the subject. With respect to a patient population, “reduce the risk of DILI” and the like means to reduce the incidence of DILI and/or the incidence of severe DILI in a given population. For example, in embodiments the present disclosure provides methods for reducing the risk of DILI in a subject in that the methods provide a means for identifying subjects who are at high risk of DILI and treating the subjects in a manner that reduces the risk that the subject will develop DILI and/or reduces the risk that the subject will develop severe or worsening DILI.

As used herein, a “therapeutically effective regimen” refers to a treatment regimen of a duration and dosage sufficient to treat a disease or condition for which a drug is prescribed.

As used herein, a “dose” refers to the dosage of a drug, for example, as indicated on the manufacture's FDA-approved or other regulatory agency-approved label for the relevant indication or indications. As used herein, the term “dosing regimen” refers to the overall therapeutic regimen of a drug. For example, a patient may be prescribed or administered a reduced dose of the drug, and/or a reduced dose over time in a reduced dosing regimen. In embodiments, the patient would not be administered, or would, in the physician's prescribed dosing regimen, be advised not to take the drug. In embodiments, the patient may have previously been prescribed or administered the drug, and the administration of the drug may be ceased temporarily or permanently in accordance with the methods provided herein.

### Drug-Induced Liver Injury (DILI)

The term “adverse drug reaction” or “ADR,” as used herein, refers to an undesired, unintended effect of a drug. ADRs can lead to severe disability or death in a subject.

Drug-induced liver injury (DILI) is one of the most common and serious ADRs, and can lead to product withdrawal post-approval. When severe, DILI causes acute liver failure, death, or a need to receive a liver transplantation for survival. In subjects with DILI, the injured cells include hepatocytes, bile duct epithelial cells, and vascular endothelial cells of the hepatic sinusoids and intrahepatic venous system. DILI can be acute or chronic, with patients suffering from acute DILI often exhibiting chronic DILI over time. The clinical manifestations of acute DILI may initially include varying elevations in the level of hepatic biochemical indexes including serum aspartate aminotransferase (AST; also called serum glutamic oxaloacetic transaminase, or SGOT), alanine aminotransferase (ALT; also called serum glutamate pyruvate transaminase or SGPT), alkaline phosphatase (ALP), and gamma-glutamyl transferase (GGT). Some patients with acute DILI may have symptoms such as fatigue, decreased appetite, aversion to oily food, tender liver, and epigastric discomfort. Patients may also exhibit jaundice, light-colored feces, and pruritus. Some acute DILI patients may have allergic manifestations including fever, rashes, increased eosinophils, and even aching pain in joints, which may be accompanied by other manifestations of extrahepatic organ damage. Some patients may develop into acute or subacute liver failure. Chronic DILI may present as, for example and without limitation, chronic hepatitis, liver fibrosis, compensated and decompensated cirrhosis, autoimmune hepatitis (AIH)-like DILI, chronic intrahepatic cholestasis, vanishing bile duct syndrome (VBDS), or a combination thereof. Patients may also present with sinusoidal obstruction syndrome (SOS)/hepatic veno-occlusive disease (VOD) or liver tumors. SOS/VOD may appear acutely with ascites, jaundice, and hepatomegaly.

DILI may be detected by monitoring levels of biochemical markers of liver function, such as AST, ALT, ALP, GGT, and bilirubin (TBil). Levels of any combination of these that are ULN (upper limit of normal) may indicate DILI. For example, DILI may be identified biochemically as reaching any of the following: (1) ALT≥3×; (2) ALT≥5×ULN; (3) ALP≥2×ULN, especially in patients with elevated 5′-nucleotidase or GGT, and without bone-diseases-related ALP elevation; (4) ALT≥3×ULN and TBil≥2×ULN (see, e.g., Aithal G P, Watkins P B, Andrade R J. “Case definition and phenotype standardization in drug-induced liver injury.” Clin Pharmacol Ther. 2011; 89(6):806-815). In some patients, a liver biopsy may be needed to determine the presence or extent of liver damage.

However, the monitoring of biochemical markers is often ineffective for reducing the risk of DILI in subjects receiving a therapeutic agent. When the drug cannot be predicted to cause liver injury in a particular patient population, DILI often goes undetected until severe damage occurs and/or progresses too fast to prevent severe damage. Thus, there is a serious need for a clinically useful method for predicting and preventing the development of DILI upon administration of a drug.

In embodiments, the present disclosure provides such methods for predicting and preventing the development of DILI. In embodiments, the present disclosure provides methods for reducing the severity of DILI and/or ameliorating the symptoms of DILI and/or reducing the time to recovery from DILI. In embodiments, the methods comprise modulating the dose of a drug provided herein that is associated with increased risk of DILI due to the presence of any one or more of SEQ ID NOs: 1-11 in the drug. Modulating the dose of a drug includes reducing the dosing level, and/or reducing the total dose administered and/or reducing the frequency of dosing. In embodiments, the methods include monitoring the subject for signs and symptoms of DILI (e.g., by monitoring the subject for elevated liver enzymes and/or bilirubin levels and/or other signs and symptoms of liver disease.

### HLA Typing

As described herein, the present Applicants have found that certain classes of patients, e.g., patients having an HLA allele provided herein, are at increased risk of developing DILI upon administration of drugs having one or more of a particular set of peptides.

The human leukocyte antigen (HLA) system describes the genetic locus encoding, among other immune-related genes, the major histocompatibility complex (MEW) proteins in humans. MHCs which are the proteins responsible for regulation of the immune system. HLA-A, HLA-B, and HLA-C are the three major antigen loci of MHC class I, which is the MEW class that presents peptides present inside the cell to CD8+ T cells. HLA-DP, HLA-DR, HLA-DQ, HLA-DM, and HLA-DO are the major loci of MHC class II, which present antigens from outside the cell to CD4+ T cells. Within these loci are pairs of alpha and beta chains: HLA-DPA1, HLA-DPB1, HLA-DRA, HLA-DRB1, HLA-DRB3, HLA-DRB4, HLA-DRB5, HLA-DQA1, HLA-DQA2, HLA-DQB1, HLA-DQB2, HLA-DMA, HLA-DMB, HLA-DOA, and HLA-DOB. The term “allele” refers herein to an alternative form of the gene at a given locus; alleles occupy the same locus on homologous chromosomes. Thus, an HLA allele is the specific allele present at a given HLA locus for an individual patient. The term “genetic variation” may be used herein to describe an HLA allele.

As used herein, “HLA typing” and the like refers to determining a subject's HLA profile, which is made up of the individual's HLA alleles. The HLA typing disclosed herein includes determining whether a particular HLA allele or set of alleles is present or absent in the patient. Various methods for HLA typing are known in the art, and any method for determining the presence or absence of HLA alleles can be used in the methods disclosed herein. HLA typing can be performed at low, intermediate, or high resolution. Low resolution HLA typing refers to typing wherein the alleles are reported at the two-digit level. An example of a representation of an HLA allele at the two-digit level is HLA-DQA1*01. Low resolution HLA typing can be achieved using serological methods. The standard method for serological HLA typing is the micro-lymphocytotoxicity assay, in which the lymphocytes of a subject are tested against a panel of antisera or monoclonal antibodies whose specificity for HLA has been previously characterized. Antibodies that bind to the HLA molecules induce lysis in the presence of complement, and indicate which HLA molecules were present on the subject's lymphocytes.

DNA- or RNA-based typing directly determines the sequence, and can provide intermediate or high resolution results. High resolution methods are preferred, in embodiments, because tight linkage disequilibrium between genes and the high degree of polymorphism present at the MHC loci can make it difficult to obtain useful information with lower resolution methods. High resolution HLA typing may be achieved using DNA- or RNA-based methods and can provide HLA reporting at the four-digit level. An example of a representation of an HLA allele at the four-digit level is HLA-DQA1*01:03. Such methods include, without limitation, PCR-sequence-specific primer (SSP) typing, PCR-sequence specific oligonucleotide (SSO) hybridization, direct sequencing, sequence based typing (SBT; e.g., Sanger sequence based method of sequencing), next generation sequencing (NGS), and the like. PCR-SSP involves allele sequence-specific primer pairs that are designed to selectively amplify target sequences that are specific to a single allele, with PCR performed in the presence of control primer pairs matching non-allelic sequences present in the sample. PCR-SSO typing uses PCR target amplification, hybridization of PCR products to a panel of immobilized sequence-specific oligonucleotides on beads, and detection of probe-bound amplification product. SBT is based on PCR target amplification, followed by sequencing of the PCR products and data analysis. NGS is an approach that can be achieved using various methods, for example those discussed in Carapito et al., Human Immunol. 77(2016) 1016-23. In principle, NGS is similar to Sanger-based sequencing, in that the bases of a DNA fragment are identified sequentially from signals emitted as each fragment is resynthesized from a DNA template strand. NGS achieves this in a scaled-up manner by allowing millions of reactions to occur in parallel. Advantages of NGS include the ability to multiplex, that is, amplify and sequence multiple HLA genes in a single reaction. Those skilled in the art will appreciate that various commercially available kits and analysis software can be used to HLA type a sample.

Intermediate resolution HLA typing refers to a situation where four-digit level typing has been completed, but there are several possibilities for the HLA present. For example, in PCR-SSP, the PCR primers used may yield more than one possible genotype that an individual may have, which may require additional testing with additional combinations of primers and/or cloning and sequencing of the clones in order to obtain an unambiguous HLA type.

In embodiments, methods for HLA typing and/or detection of HLA alleles are referred to as “genetic assays.” Genetic assays can be used to detect the presence or absence of one or more genetic variations (e.g., one or more alleles). For example, in embodiments, the present disclosure provides methods for treating a subject in need of a therapy for a disease or condition, such as an autoimmune disease or cancer, comprising testing the subject for the presence or absence of one or more genetic variations, using a genetic assay. In embodiments, the genetic variation comprises one or more of alleles HLA-B*39:01, HLA-C*12:03, HLA-DRB1*03:01, HLA-DQB1*02:01, and HLA-DQA1*03:03. In embodiments, the genetic variation comprises the HLA-B*39:01 allele.

In an aspect, the present inventors found that a particular HLA allele or one of a set of particular HLA alleles was associated with the development of DILI after subjects were administered infliximab. The inventors undertook an extensive statistical analysis of the HLA alleles present in samples from subjects who experienced DILI following administration with infliximab, as compared to samples from healthy subjects (i.e., subjects who received infliximab and did not experience DILI) and found a statistically significant increased risk of developing DILI associated with HLA-B*39:01. In embodiments, additional alleles associated with significant risk of developing DILI were identified and include HLA-C*12:03, HLA-DRB1*03:01, HLA-DQB1*02:01, and HLA-DQA1*03:03.

The inventors further found that certain peptides present in the infliximab amino acid sequence interact with HLA-B*39:01. Without wishing to be bound by theory, the interaction of the infliximab peptide sequences with HLA-B*39:01 can cause DILI or lead to the development of DILI or exacerbate DILI in the subject. Moreover, the inventors determined that drugs other than infliximab have one or more peptides that interact with HLA-B*39:01. This peptide-HLA-B*39:01 interaction is believed, without wishing to be bound by theory, to be the trigger for development of DILI in subjects administered a therapeutic agent comprising one or more of the peptides. In embodiments, the peptides present on certain therapeutic agents that interact with HLA-B*39:01 include SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or any combination thereof. To elicit DILI, the therapeutic agent may have one, two, three, four, five, six, seven, eight, nine, ten, or all eleven of SEQ ID NOs: 1-11.

### Peptides

The present disclosure provides methods for treating diseases and disorders in subjects comprising the administration of a drug that has been identified as either having or not having one or more of a particular set of peptide sequences. In embodiments, the peptide sequences are SEQ ID NO: 1 (THQGLSSPV), SEQ ID NO: 2 (TKGPSVFPL), SEQ ID NO: 3 (QSHSWPF), SEQ ID NO: 4 (IRSKSIN), SEQ ID NO: 5 (YSLSSTLTL), SEQ ID NO: 6 (TQSPAILSV), SEQ ID NO: 7(STYSLSSTL), SEQ ID NO: 8 (VQWKVDNAL), SEQ ID NO: 9 (CQQSHSWPF), SEQ ID NO: 10 (LQSSGLYSL), and SEQ ID NO: 11(STSGGTAAL).

In embodiments, the drug has any one or more of SEQ ID NOs: 1-11. In embodiments of the present disclosure, the methods provided relate to reducing the risk of DILI and/or treating a subject or patient population with a drug that has any one of SEQ ID NOs: 1-11, or a drug that has one or more of SEQ ID NOs: 1-11, for example, a drug that has any one or more of SEQ ID NOs: 1, 2, and 5-11; or a drug that has SEQ ID NO: 1 or SEQ ID NO: 2, or a drug that has SEQ ID NO: 1 and SEQ ID NO: 2. In embodiment, the methods provided relate to reducing the risk of DILI and/or treating a subject or patient population, wherein the subject or patient population is not administered any drug that comprises any one of SEQ ID NOs: 1-11; for example, the subject or patient population is not administered a drug that has any one or more of SEQ ID NOs: 1, 2, and 5-11; or a drug that has SEQ ID NO: 1 or SEQ ID NO: 2, or a drug that has SEQ ID NO: 1 and SEQ ID NO: 2. Exemplary drugs and the presence or absence of the peptides are provided below in Tables 1A and 1B.

TABLE 1A
Exemplary drugs and the presence or absence of SEQ ID NO: 1-5

| | | THQGLSSPV | TKGPSVFPL | QSHSWPF | IRSKSIN | YSLSSTLTL |
|---|---|---|---|---|---|---|
| | Mechanism of | SEQ ID NO: | SEQ ID NO: | SEQ ID NO: | SEQ ID | SEQ ID NO: |
| Drug | Action | 1 | 2 | 3 | NO: 4 | 5 |
| Adalimumab | Tumor necrosis | Y | Y | N | N | Y |
| | factor (TNF) | | | | | |
| | blocker | | | | | |
| Belimumab | B-lymphocyte | N | Y | N | N | N |
| | stimulator | | | | | |
| | (BLyS)-specific | | | | | |
| | inhibitor | | | | | |
| Canakinumab | Interleukin-1 | Y | Y | N | N | Y |
| | Beta blocker | | | | | |
| Certolizumab | Tumor necrosis | Y | Y | N | N | Y |
| | factor (TNF) | | | | | |
| | blocker | | | | | |
| Daclizumab | Interleukiin-2 | Y | Y | N | N | Y |
| | receptor blocker | | | | | |
| Efalizumab | anti-CD11a | Y | Y | N | N | Y |
| | antibody | | | | | |
| Infliximab | Tumor necrosis | Y | Y | Y | Y | Y |
| | factor (TNF) | | | | | |
| | blocker | | | | | |
| Ixekizumab | Interleukin-17A | Y | Y | N | N | Y |
| | antagonist | | | | | |
| Rituximab | CD20 antibody | Y | Y | N | N | Y |
| Ustekinumab | integrin | Y | Y | N | N | Y |
| | receptor | | | | | |
| | antagonist | | | | | |
| Natalizumab | integrin | Y | Y | N | N | Y |
| | receptor | | | | | |
| | antagonist | | | | | |
| Vedolizumab | IL-6 receptor | Y | Y | N | N | Y |
| | antagonist | | | | | |
| Tocilizumab | TNF blocker | Y | Y | N | N | Y |
| Secukinumab | IL-6 receptor | Y | Y | N | N | Y |
| | antagnoist | | | | | |
| Sarilumab | CD52 antibody | Y | Y | N | N | Y |
| Alemtuzumab | IL-17RA | Y | Y | N | N | Y |
| | antagonist | | | | | |
| Brodalumab | IL-23 | Y | Y | N | N | Y |
| | antagonist | | | | | |
| Guselkumab | IL-23 | N | N | N | N | N |
| | antagonist | | | | | |
| Obinutuzumab | CD20 antibody | Y | Y | N | N | Y |
| Ofatumumab | CD20 antibody | Y | Y | N | N | Y |
| Ibritumomab | CD20 | Y | N | N | N | Y |
| | radiotherapeutic | | | | | |
| | antibody | | | | | |
| Abciximab | Glycoprotein | Y | Y | N | N | Y |
| | IIb/IIIa receptor | | | | | |
| | antagonist | | | | | |
| Atezolizumab | PD-L1 antibody | Y | Y | N | N | Y |
| Avelumab | PD-L1 antibody | Y | Y | N | N | Y |
| Basiliximab | CD25 antibody | Y | Y | N | N | Y |
| Benralizumab | CD125 | Y | Y | N | N | Y |
| | antibody | | | | | |
| Bevacizumab | VEGF-A | Y | Y | N | N | Y |
| | antibody | | | | | |
| Bezlotoxumab | C. difficile toxin | Y | Y | N | N | Y |
| | B antibody | | | | | |
| Blinatumomab | CD19/CD3 bi- | N | N | N | N | N |
| | specific T cell | | | | | |
| | engager | | | | | |
| Burosumab | FGF23 | Y | Y | N | N | N |
| | antibody | | | | | |
| Caplacizumab | Von Willebrand | N | N | N | N | N |
| | factor (VWF) | | | | | |
| | single domain | | | | | |
| | antibody | | | | | |
| Cetuximab | EGFR antibody | Y | Y | N | N | Y |
| Daratumumab | CD38 antibody | Y | Y | N | N | Y |
| Denosumab | RANKL | V | Y | N | N | Y |
| | antibody | | | | | |
| Dupilumab | IL-4Rα | Y | Y | N | N | Y |
| | antibody | | | | | |
| Durvalumab | PD-L1 antibody | Y | Y | N | N | Y |
| Eculizumab | C5 antibody | Y | Y | N | N | Y |
| Elotuzumab | SLAMF7 | Y | Y | N | N | Y |
| | antibody | | | | | |
| Emicizumab | Factor IX, | N | Y | N | N | N |
| | factor X | | | | | |
| | antibody | | | | | |
| Erenumab | CGRPR | N | Y | N | N | N |
| | antibody | | | | | |
| Fremanezumab | CGRP antibody | Y | Y | N | N | Y |
| Galcanezumab | Calcitonin | Y | Y | N | N | Y |
| | antibody | | | | | |
| Gemtuzumab | CD33 antibody | Y | Y | N | N | Y |
| ozogamicin | | | | | | |
| Golimumab | TNFα antibody | N | N | N | N | N |
| Ibalizumab | CD4 antibody | Y | Y | N | N | Y |
| Idarucizumab | Dabigatran | Y | Y | N | N | Y |
| | antibody | | | | | |
| Ipilimumab | CD152 | Y | Y | N | N | Y |
| | antibody | | | | | |
| Lanadelumab | Kallikrein | Y | Y | N | N | Y |
| | antibody | | | | | |
| Moxetumomab | CD22 antibody | N | N | N | N | N |
| pasudotox-tdfk | | | | | | |
| Necitumumab | EGFR antibody | Y | Y | N | N | Y |
| Nivolumab | PD-1 antibody | Y | Y | N | N | Y |
| Olaratumab | PDGFRα | Y | Y | N | N | Y |
| | antibody | | | | | |
| Omalizumab | IgE antibody | Y | Y | N | N | Y |
| Palivizumab | RSV F protein | N | N | N | N | N |
| | antibody | | | | | |
| Panitumumab | EGFR antibody | Y | Y | N | N | Y |
| Pembrolizumab | PD-1 antibody | Y | Y | N | N | Y |
| Pertuzumab | Her2/neu | y | Y | N | N | Y |
| | antibody | | | | | |
| Ramucirumab | VEGFR2 | Y | Y | N | N | Y |
| | antibody | | | | | |
| Ranibizumab | VEGF-A | Y | Y | N | N | Y |
| | antibody | | | | | |
| Ravulizumab | C5 antibody | Y | Y | N | N | Y |
| Risankizumab | IL23A antibody | Y | Y | N | N | y |
| Siltuximab | IL-6 antibody | Y | Y | N | N | Y |
| Tildrakizumab | IL-23 antibody | N | N | N | N | N |
| Tositumomab | CD20 antibody | Y | N | N | N | Y |
| Trastuzumab | Her2/neu | Y | Y | N | N | Y |
| | antibody | | | | | |

Y indicates that the indicated sequence is present in the indicated drug;
N indicates that the indicated sequence is not present in the indicated drug.

TABLE 1B
Exemplary drugs and presence or absence of SEQ ID NOs: 6-11

| | TQSPAILSV | STYSLSSTL | VQWKVDNAL | CQQSHSWPF | LQSSGLYSL | STSGGTAAL |
|---|---|---|---|---|---|---|
| | SEQ ID NO: | SEQ ID NO: | SEQ ID NO: | SEQ ID NO: | SEQ ID NO: | SEQ ID NO: |
| Drug | 6 | 7 | 8 | 9 | 10 | 11 |
| Adalimumab | N | Y | Y | N | Y | Y |
| Belimumab | N | N | N | N | Y | Y |
| Canakinumab | N | Y | Y | N | Y | Y |
| Certolizumab | N | Y | Y | N | Y | Y |
| Daclizumab | N | Y | Y | N | Y | Y |
| Efalizumab | N | Y | Y | N | Y | Y |
| Infliximab | Y | Y | Y | Y | Y | Y |
| Ixekizumab | N | Y | Y | N | Y | Y |
| Rituximab | N | Y | Y | N | Y | Y |
| Ustekinumab | N | Y | Y | N | Y | Y |
| Natalizumab | N | Y | Y | N | Y | N |
| Vedolizumab | N | Y | Y | N | Y | Y |
| Tocilizumab | N | Y | Y | N | Y | Y |
| Secukinumab | N | Y | Y | N | Y | Y |
| Sarilumab | N | Y | Y | N | Y | Y |
| Alemtuzumab | N | Y | Y | N | Y | Y |
| Brodalumab | N | Y | Y | N | Y | N |
| Guselkumab | N | N | N | N | Y | Y |
| Obinutuzumab | N | Y | Y | N | Y | Y |
| Ofatumumab | N | Y | Y | N | Y | N |
| Ibritumomab | N | Y | Y | N | N | N |
| Abciximab | N | Y | Y | N | Y | Y |
| Atezolizumab | N | Y | Y | N | Y | Y |
| Avelumab | N | N | N | N | N | N |
| Basiliximab | N | Y | Y | N | Y | Y |
| Benralizumab | N | Y | Y | N | Y | Y |
| Bevacizumab | N | Y | Y | N | Y | Y |
| Bezlotoxumab | N | Y | Y | N | Y | Y |
| Blinatumomab | N | N | N | N | N | N |
| Burosumab | N | Y | Y | N | Y | Y |
| Caplacizumab | N | N | N | N | N | N |
| Cetuximab | N | Y | Y | N | Y | Y |
| Daratumumab | N | Y | Y | N | Y | Y |
| Denosumab | N | Y | Y | N | Y | Y |
| Dupilumab | N | Y | Y | N | Y | N |
| Durvalumab | N | Y | Y | N | Y | Y |
| Eculizumab | N | Y | Y | N | Y | Y |
| Elotuzumab | N | Y | Y | N | Y | Y |
| Emicizumab | N | N | Y | N | Y | N |
| Erenumab | N | N | N | N | Y | Y |
| Fremanezumab | N | Y | Y | N | Y | N |
| Galcanezumab | N | Y | Y | N | Y | N |
| Gemtuzumab | N | Y | Y | N | Y | N |
| ozogamicin | | | | | | |
| Golimumab | N | N | N | N | N | N |
| Ibalizumab | N | Y | Y | N | Y | N |
| Idarucizumab | N | Y | Y | N | Y | Y |
| Ipilimumab | N | Y | Y | N | Y | Y |
| Lanadelumab | N | Y | Y | N | Y | Y |
| Moxetumomab | N | N | N | N | N | N |
| pasudotox | | | | | | |
| Necitumumab | N | Y | Y | N | Y | N |
| Nivolumab | N | Y | Y | N | Y | N |
| Olaratumab | N | Y | Y | N | Y | Y |
| Omalizumab | N | Y | Y | N | Y | Y |
| Palivizumab | N | N | N | N | N | N |
| Panitumumab | N | Y | Y | N | Y | N |
| Pembrolizumab | N | Y | Y | N | Y | N |
| Pertuzumab | N | Y | Y | N | Y | Y |
| Ramucirumab | N | Y | Y | N | Y | Y |
| Ranibizumab | N | Y | Y | N | Y | Y |
| Ravulizumab | N | Y | Y | N | Y | N |
| Risankizumab | N | Y | Y | N | Y | Y |
| Siltuximab | N | Y | Y | N | Y | Y |
| Tildrakizumab | N | N | N | N | N | N |
| Tositumomab | N | Y | Y | N | Y | Y |

Y indicates that the indicated sequence is present in the indicated drug;
N indicates that the indicated sequence is not present in the indicated drug.

### Drugs

The present disclosure provides methods for treating diseases and disorders in subjects comprising the administration of a drug that has been identified as either having or not having a particular peptide sequence (e.g., one or more of SEQ ID NOs: 1-11).

In embodiments, the drug is selected from the group consisting of abciximab, adalimumab, alemtuzumab, alirocumab, atezolizumab, atoltivimab, maftivimab, odesivimab, avelumab, basiliximab, belantamab mafodotin, belimumab, benralizumab, bevacizumab, bezlotoxumab, blinatumomab, brentuximab vedotin, brodalumab, brolucizumab, burosumab, canakinumab, caplacizumab, capromab pendetide, cemiplimab, certolizumab pegol, cetuximab, crizanlizumab, daclizumab, daratumumab, denosumab, dinutuximab, dupilumab, durvalumab, eculizumab, efalizumab, elotuzumab, emapalumab, emicizumab, enfortumab vedotin, erenumab, evolocumab, fanolesomab, fremanezumab, galcanezumab, gemtuzumab ozogamicin, golimumab, guselkumab, ibalizumab, ibritumomab tiuxetan, idarucizumab, inebilizumab, infliximab, inotuzumab ozogamicin, ipilimumab, isatuximab, ixekizumab, lanadelumab, mepolizumab, mogamulizumab, moxetumomab pasudotox, natalizumab, naxitamab, necitumumab, nivolumab, nofetumomab, obiltoxaximab, obinutuzumab, ocrelizumab, ofatumumab, olaratumab, omalizumab, palivizumab, panitumumab, pembrolizumab, pertuzumab, polatuzumab vedotin, ramucirumab, ranibizumab, ravulizumab, raxibacumab, reslizumab, risankizumab, rituximab, romosozumab, sacituzumab govitecan, sarilumab, satralizumab, secukinumab, siltuximab, tafasitamab, teprotumumab, tildrakizumab, tocilizumab, tositumomab, trastuzumab, ustekinumab, vedolizumab, tisotumab vedotin, epcoritamab, amivantamab, teclistamab, camidanlumab tesirine, talquetamab, actimab-a, lintuzumab.

Infliximab is a mouse-human chimeric monoclonal antibody that binds to human tumor necrosis factor (TNF). The amino acid sequence of infliximab includes all of SEQ ID NOs: 1-11. Due to the importance of TNF in inflammatory processes, this inhibitory antibody has a potent anti-inflammatory effect. Infliximab has been approved for use in the United States for rheumatoid arthritis, ankylosing spondylitis, psoriatic arthritis, plaque psoriasis, Crohn's disease (adult and pediatric) and ulcerative colitis (adult and pediatric). Infliximab can be indicated for the treatment of moderate to severe active rheumatoid arthritis in patients who have had inadequate response or tolerance to methotrexate. Infliximab is also approved and/or under clinical investigation for use in various other diseases and disorders, including, but not limited to, refractory uveoretinitis associated with Behcet's disease, pustular psoriasis, erythrodermic psoriasis; intestinal-Behcet's disease, neuro-Behcet's disease, vasculo-Behcet's disease, sterile corneal melt, depression, bipolar depression (e.g., bipolar I/II depression), refractory intestinal Behcet's disease, Takayasu's arteritis, dolichoectatic vertibrobasilar (DVB) aneurysms, refractory idiopathic scleritis, graft versus host disease (GVHD), steroid refractory acute graft versus host disease, macular edema, diabetic macular edema, refractory polymyalgia rheumatic (PMR), choroidal neovascularization, Kawasaki disease, melanoma, advanced melanoma, inflammatory skin disease, sarcoidosis, and anal fistulae.

Infliximab is marketed under the trade name REMICADE®, and the FDA and European Medicines Agency (EMA) have also approved an infliximab biosimilar, INFLECTRA®. Various other biosimilars are also approved in some countries outside the US and/or are in development worldwide.

The dosing for infliximab varies by indication, but generally is administered intravenously at a dose of 5 mg/kg, and over several doses (e.g., at 1, 2, and 6 weeks, with a maintenance dose at 6 or 8 week intervals thereafter). In embodiments, a therapeutically effective amount of infliximab is at least 5 mg/kg infliximab. For some patients, the prescribed dose may be increased to 10 mg/kg. For some patients with rheumatoid arthritis, infliximab may be administered in conjunction with methotrexate, and the prescribed dose of infliximab may be 3 mg/kg, with or without an increase to 10 mg/kg and/or a maintenance dose every 4, 6, or 8 weeks. Infliximab has been known to cause hepatotoxicity, and even severe hepatic reactions that can be fatal or necessitate a liver transplant. Generally, when a patient receiving infliximab exhibits jaundice and/or liver enzyme elevations, administration of infliximab is ceased. For some patients, corticosteroid treatment is needed once liver injury has presented. Subjects experiencing liver toxicity may be switched to an alternative therapeutic agent, such as etanercept, or other alternatives to infliximab.

Adalimumab is a TNF blocker that is marketed under the trade name HUMIRA®, among others. Like infliximab, adalimumab is a potent anti-inflammatory antibody. Adalimumab's approved uses include rheumatoid arthritis, polyarticular juvenile idiopathic arthritis, psoriatic arthritis, ankylosing spondylitis, Crohn's disease, plaque psoriasis; hidradenitis suppartiva, panuveitis, intestinal Behcet's disease, pyoderma Gangrenosum, and axial spondyloarthritis including ankylosing spondylitis and when there are clear signs of inflammation but X-ray does not show disease. Adalimumab is generally administered parenterally, such as by intramuscular, subcutaneous, or intravenous injection; for example, adalimumab's approved label includes administration by subcutaneous injection of about 40 mg every other week, or about 80 mg or about 160 mg as an initial dose followed by 40 mg maintenance doses. There have been reports of severe hepatic reactions including acute liver failure in patients receiving TNF blockers. Adalimumab's amino acid sequence includes SEQ ID NOs: 1, 2, 5, 7, 8, 10, and 11.

Belimumab (BENLYSTA®) is a B-lymphocyte stimulator (BLyS)-specific inhibitor that is used to treat systemic lupus erythematosus. BENLYSTA is administered intravenously or subcutaneously. For intravenous administration, dosing is 10 mg/kg at 2 week intervals for the first 3 doses and at 4 week intervals thereafter. Premedication for prophylaxis against infusion reactions and hypersensitivity reactions may be considered. For subcutaneous administration, dosing is 200 mg once weekly. The dosage form for intravenous infusion is 120 mg or 400 mg lyophilized powder in a single dose vial for reconstitution and dilution. The dosage form for subcutaneous injection is 200 mg/mL in a single dose prefilled auto injector or prefilled syringe. Belimumab's amino acid sequence includes SEQ ID NOs: 2, 10, and 11.

Canakinumab (ILARIS®) is an antibody that blocks interleukin-10. The antibody sequence includes SEQ ID NOs: 1, 2, 5, 7, 8, 10, and 11. Canakinumab is used for the treatment of various diseases including Cryopyrin-Associated Periodic Syndromes (CAPS) including Familial Cold Autoinflammatory Syndrome (FCAS) and Muckle-Wells Syndrome (MWS); Tumor Necrosis Factor Receptor Associated Periodic Syndrome (TRAPS); Hyperimmunoglobulin D Syndrome (HIDS)/Mevalonate Kinase Deficiency (MKD); Familial Mediterranean Fever (FMF); Systemic Juvenile Idiopathic Arthritis (SJIA); Still's disease; gouty arthritis; and neonatal-onset multisystem inflammatory disease. Dosing is 150 mg for CAPS patients with body weight greater than 40 kg and 2 mg/kg for CAPS patients with body weight greater than or equal to 15 kg and less than or equal to 40 kg. For children 15 to 40 kg with an inadequate response, the dose can be increased to 3 mg/kg. Administer subcutaneously every 8 weeks. For TRAPS, HIDS/MKD, and FMF, dosing is as follows: for patients with body weight less than or equal to 40 kg, the recommended starting dose is 2 mg/kg every 4 weeks, which can be increased to 4 mg/kg every 4 weeks if the clinical response is not adequate. For patients with body weight greater than 40 kg, the recommended starting dose is 150 mg every 4 weeks, which can be increased to 300 mg every 4 weeks if the clinical response is not adequate. For Still's disease (AOSD and SJIA), dosing is 4 mg/kg (with a maximum of 300 mg) for patients with a body weight greater than or equal to 7.5 kg, subcutaneously every 4 weeks. For injection, the dosing form is 150 mg lyophilized powder in single-dose vials for reconstitution or 150 mg/mL solution in single-dose vials.

Certolizumab (CIMZIA® and its biosimilars) is an anti-TNF antibody used for the treatment of autoimmune diseases, such as Crohn's disease, rheumatoid arthritis, psoriatic arthritis, ankylosing spondylitis, non-radiographic axial spondyloarthritis, and plaque psoriasis. Certolizumab's approved dosing regimens include administration at a dose of 400 mg, administered in two subcutaneous injections of 200 mg; or an initial 400 mg dose followed by doses of 200 mg or 400 mg weekly or every other week after the initial dose. Certolizumab may be administered parenterally, such as by intramuscular, subcutaneous, or intravenous injection. The dosage form is 200 mg lyophilized powder in a single dose vial or 200 mg/mL solution in a single dose prefilled syringe. Elevated liver enzymes and hepatitis have been reported. The amino acid sequence of the antibody includes SEQ ID NOs: 1, 2, 5, 7, 8, 10, and 11.

Daclizumab (ZINBRYTA®) is a CD25 (IL-2 receptor)-specific antibody used to treat multiple sclerosis or other autoimmune disorders. The sequence of daclizumab includes SEQ ID NOs: 1, 2, 5, 7, 8, 10, and 11. Administration is subcutaneous injection of 150 mg once monthly. Prior to initiating ZINBRYTA, patient serum transaminases (alanine aminotransferase (ALT) and aspartate aminotransferase (AST)) and total bilirubin levels may be obtained. Initiation of ZINBRYTA is contraindicated in patients with pre-existing hepatic disease or hepatic impairment including ALT or AST at least 2 times the ULN. Interruption or discontinuation is recommended for management of certain liver test abnormalities. The dosage forms are 150 mg/mL solution in a single-dose prefilled auto injector or in a single-dose prefilled syringe. ZINBRYTA can cause severe liver injury, including autoimmune hepatitis and liver failure and is contraindicated in patients with pre-existing hepatic disease or hepatic impairment, including ALT or AST at least 2 times the ULN and/or history of autoimmune hepatitis or other autoimmune condition involving the liver.

Efalizumab (RAPTIVA®) is an antibody that is specific for CD11a. It has been used to treat autoimmune disorders such as moderate to severe chronic plaque psoriasis. RAPTIVA is administered in a single 0.7 mg/kg conditioning dose followed by weekly doses of 1 mg/kg, with maximum single dose not to exceed a total of 200 mg. SEQ ID NOs: 1, 2, 5, 7, 8, 10, and 11 are present in the sequence of efalizumab.

Ixekizumab (TALTZ®) is an IL-17a antagonist antibody that includes SEQ ID NOs: 1, 2, 5, 7, 8, 10, and 11. Ixekizumab is used to treat diseases such as moderate to severe plaque psoriasis; active psoriatic arthritis; active ankylosing spondylitis; psoriasis vulgaris, pustular psoriasis, and erythrodermic psoriasis. For adult plaque psoriasis, the dose is 160 mg (two 80 mg injections) at week 0, followed by 80 mg at weeks 2, 4, 6, 8, 10, and 12, then 80 mg every 4 weeks. For pediatric plaque psoriasis, in patients weighing greater than 50 kg, the recommended dose is 160 mg (two 80 mg injections) at week 0, followed by 80 mg every 4 weeks; for patients weighing 25-50 kg, the recommended dose is 80 mg atwWeek 0, followed by 40 mg every 4 weeks; and for patients weighing less than 25 kg, the recommended dose is 40 mg at week 0, followed by 20 mg every 4 weeks. For psoriatic arthritis, the recommended dose is 160 mg by subcutaneous injection (two 80 mg injections) at week 0, followed by 80 mg every 4 weeks. For psoriatic arthritis patients with coexistent moderate-to-severe plaque psoriasis, the same dosing regimen as that for adult plaque psoriasis is used. For ankylosing spondylitis, the recommended dose is 160 mg by subcutaneous injection (two 80 mg injections) at week 0, followed by 80 mg every 4 weeks. For non-radiographic axial spondyloarthritis, the recommended dose is 80 mg by subcutaneous injection every 4 weeks. TALTZ may be administered alone or in combination with a conventional DMARD (e.g., methotrexate). The dosage form for administration via an autoinjector is an 80 mg/mL solution for injection in a single-dose prefilled autoinjector. The dosage form for the prefilled syringe is a 80 mg/mL solution for injection in a single-dose prefilled syringe.

Rituximab and rituximab/hyaluronidase (RITUXAN® and RITUXAN HYCELA®, respectively) are used to treat various autoimmune diseases and cancers including non-Hodgkin's Lymphoma (NHL), CD20-positive NHL; chronic lymphocytic leukemia (CLL); rheumatoid arthritis; granulomatosis with polyangitis (Wegner's granulomatosis); microscopic polyangitis; moderate to severe pemphigus vulgaris; follicular lymphoma; diffuse large B-cell lymphoma; Refractory nephrotic syndrome. Rituximab is an antibody that binds to CD20 on B cells and includes SEQ ID NOs: 1, 2, 5, 7, 8, 10, and 11. Rituximab's approved label includes parenteral administration in a dose of about 250 mg/m2, about 275 mg/m2, or about 500 mg/m2; or a dose of about 500 mg, about 1000 mg, or about 1500 mg. For example, rituximab may be administered to rheumatoid arthritis patients in combination with methotrexate; and may be administered to rheumatoid arthritis patients in courses of two 1000 mg infusions, each course separated by 16, 18, 20, 22, 24, or more weeks.

Ustekinumab (STELARA® and its biosimilars) is an IL-12 and IL-23 antagonist used to treat, e.g., active psoriatic arthritis, moderate to severe plaque psoriasis, moderate to severe Crohn's disease, and moderate to severe ulcerative colitis. The sequence of this antibody includes SEQ ID NOs: 1, 2, 5, 7, 8, 10, and 11. Ustekinumab may be administered parenterally, such as by intramuscular, subcutaneous, or intravenous injection, in a dose of about 45 mg, about 90 mg, about 260 mg, about 390 mg, or about 520 mg. For example, approved uses of ustekinumab include subcutaneous administration every 4 weeks, every 8 weeks or every 12 weeks; or intravenous infusion followed by subcutaneous doses.

Natalizumab (Tysabri®) is an integrin receptor antagonist indicated for multiple sclerosis (MS; monotherapy for the treatment of relapsing forms of MS, to include clinically isolated syndrome, relapsing-remitting disease, and active secondary progressive disease, in adults) and Crohn's disease (for inducing and maintaining clinical response and remission in adult patients with moderately to severely active Crohn's disease with evidence of inflammation who have had inadequate response to, or are unable to tolerate, conventional therapies and inhibitors of TNFα). TYSABRI may be administered intravenously over one hour, at a dose of 300 mg, every four weeks. The dosage form is a 300 mg/15 mL (20 mg/mL) solution. Significant liver injury, including liver failure requiring transplant, has occurred in patients and TYSABRI may be discontinued in patients with evidence of liver injury. Signs of liver injury, including markedly elevated serum hepatic enzymes and elevated total bilirubin, occurred as early as six days after the first dose; signs of liver injury have also been reported for the first time after multiple doses. In some patients, liver injury recurred upon rechallenge, providing evidence that TYSABRI caused the injury. The combination of transaminase elevations and elevated bilirubin without evidence of obstruction is generally recognized as an important predictor of severe liver injury that may lead to death or the need for a liver transplant in some patients. TYSABRI should be discontinued in patients with jaundice or other evidence of significant liver injury. The sequence of natalizumab includes SEQ ID NOs: 1, 2, 5, 7, 8, and 10.

Vedolizumab (Entyvio®) is an integrin receptor antagonist indicated for use in moderately to severely active ulcerative colitis or Crohn's disease. The recommended dosage is 300 mg infused intravenously over approximately 30 minutes at zero, two, and six weeks, and then every eight weeks thereafter. There have been reports of elevations of transaminase and/or bilirubin in patients receiving ENTYVIO. The combination of transaminase elevations and elevated bilirubin without evidence of obstruction is generally recognized by clinicians as an important predictor of severe liver injury that may lead to death or the need for a liver transplant in some patients. ENTYVIO should be discontinued in patients with jaundice or other evidence of significant liver injury. The sequence of vedolizumab includes SEQ ID NOs: 1, 2, 5, 7, 8, 10, and 11.

Tocilizumab (Actemra®) is an interleukin-6 (IL-6) receptor antagonist indicated for Rheumatoid Arthritis (RA; adult patients with moderately to severely active rheumatoid arthritis who have had an inadequate response to one or more Disease-Modifying Anti-Rheumatic Drugs (DMARDs)), Giant Cell Arteritis (GCA; adult patients), Polyarticular Juvenile Idiopathic Arthritis (PJIA; patients 2 years of age and older with active polyarticular juvenile idiopathic arthritis), Systemic Juvenile Idiopathic Arthritis (SJIA; patients 2 years of age and older with active systemic juvenile idiopathic arthritis), and Cytokine Release Syndrome (CRS; adults and pediatric patients 2 years of age and older with chimeric antigen receptor (CAR) T cell-induced severe or life-threatening cytokine release syndrome. For RA, pJIA and sJIA, Actemra may be used alone or in combination with methotrexate: and in RA, other DMARDs may be used. The recommended adult intravenous dose, when used in combination with DMARDs or as monotherapy, is 4 mg per kg every 4 weeks followed by an increase to 8 mg per kg every 4 weeks based on clinical response. Doses exceeding 800 mg per infusion are not recommended. The recommended dose for adults and subcutaneous doses is by weight: for patients less than 100 kg, 162 mg administered subcutaneously every weight other week, followed by an increase to every week based on clinical response; for patients at or above 100 kg, 162 mg administered subcutaneously every week. For GCA, the recommended adult subcutaneous dosage is 162 mg given once every week in combination with a tapering course of glucocorticoids. A dose of 162 mg given once every other week in combination with a tapering course of glucocorticoids may be prescribed based on clinical considerations. ACTEMRA can be used alone following discontinuation of glucocorticoids. For PJIA, the recommended intravenous dosage is by weight: for patients less than 30 kg weight, 10 mg per kg every 4 weeks; for patients at or above 30 kg weight, 8 mg per kg every 4 weeks. For PJIA, the recommended subcutaneous dosage is also by weight: for patients less than 30 kg weight, 162 mg once every three weeks; for patients at or above 30 kg weight, 162 mg once every two weeks. For SJIA, the recommended intravenous dosage for patients less than 30 kg weight is 12 mg per kg every 2 weeks; the recommended intravenous dosage for patients at or above 30 kg weight is 8 mg per kg every 2 week; the recommended subcutaneous dosage for patients less than 30 kg weight is 162 mg every two weeks; and the recommended subcutaneous dosage for patients at or above 30 kg weight is 162 mg every week. For CRS, the intravenous dosage for patients less than 30 kg weight is 12 mg per kg every 2 weeks, and the recommended intravenous dosage for patients at or above 30 kg weigh tis 8 mg per kg every 2 weeks; doses exceeding 800 mg per infusion are not recommended. For liver enzyme abnormalities, doses may be modified. For patients receiving intravenously with persistent increases, reduce dose to 4 mg per kg or hold Acterma until ALT or AST have normalized. For patients receiving subcutaneously with persistent increases, reduce injection frequency to every other week or hold dosing until ALT or AST have normalized. Resume at every other week and increase frequency to every week as clinically appropriate. Where a patient exhibits greater than 3 to 5×ULN (confirmed by repeat testing), Acterma should be held until than 3×ULN; and follow recommendations above for greater than 1 to 3×ULN. For persistent increases greater than 3×ULN, discontinue the drug. For Low Absolute Neutrophil Count—ANC (cells per mm³): For ANC 500 to 1000, hold Acterma dosing and when ANC greater than 1000: for patients receiving intravenously, resume at 4 mg per kg and increase to 8 mg per kg as clinically appropriate; for patients receiving subcutaneously, resume at every other week and increase frequency to every week as clinically appropriate. For ANC less than 500, discontinue ACTEMRA. For low platelet count (cells per mm³) of 50,000 to 100,000 hold ACTEMRA dosing. When platelet count is greater than 100,000, for patients receiving intravenously, resume at 4 mg per kg and increase to 8 mg per kg as clinically appropriate; for patients receiving subcutaneously, resume Acterma at every other week and increase frequency to every week as clinically appropriate; for less than 50,000, discontinue ACTEMRA. The dosage form for intravenous infusion of ACTEMRA is 80 mg/4 mL (20 mg/mL), 200 mg/10 mL (20 mg/mL), or 400 mg/20 mL (20 mg/mL) in single-dose vials for further dilution. The dosage form for subcutaneous administration is 162 mg/0.9 mL in a single-dose prefilled syringe or single-dose prefilled ACTPen® auto injector. Patients should be monitored for signs and symptoms of hepatic injury and Acterma should be modified or discontinued if abnormal liver tests persist or worsen or if clinical signs and symptoms of liver disease develop. Serious cases of hepatic injury have been observed in patients taking intravenous or subcutaneous ACTEMRA. Some of these cases have resulted in liver transplant or death. Time to onset for cases ranged from months to years after treatment initiation with tocilizumab. While most cases presented with marked elevations of transaminases (>5 times ULN), some cases presented with signs or symptoms of liver dysfunction and only mildly elevated transaminases. For RA and GCA patients, clinicians may obtain a liver test panel (ALT, AST, alkaline phosphatase, and total bilirubin) before initiating Acterma, every 4 to 8 weeks after start of therapy for the first 6 months of treatment and every 3 months thereafter. It is not recommended to initiate ACTEMRA treatment in RA or GCA patients with elevated transaminases ALT or AST greater than 1.5×ULN. A similar pattern of liver enzyme elevation is noted with ACTEMRA treatment in the PJIA and SJIA populations. Liver test panel may be monitored at the time of the second administration and thereafter every 4 to 8 weeks for PJIA and every 2 to 4 weeks for SJIA. The sequence of toclilizumab includes SEQ ID NOs: 1, 2, 5, 7, 8, 10, and 11.

Golimumab (SYOY) (Simponi®, Simponi Aria®) is a TNF blocker indicated for moderately to severely active rheumatoid arthritis (RA) in combination with methotrexate, active psoriatic arthritis (PsA) alone, or in combination with methotrexate, active ankylosing spondylitis (AS), and moderate to severe Ulcerative colitis (UC) with an inadequate response or intolerant to prior treatment or requiring continuous steroid therapy. For RA, PsA, and AS, 50 mg is administered by subcutaneous injection once a month. For RA, SIMPONI should be given in combination with methotrexate; for PsA or AS, SIMPONI may be given without methotrexate or other nonbiologic DMARDs. For RA, PsA, or AS, corticosteroids, DMARDs, and/or NSAIDs may be continued during treatment with SIMPONI. SIMPONI ARIA administration is 2 mg/kg intravenous infusion over 30 minutes at weeks 0 and 4, then every 8 weeks. The dosage forms for SIMPONI are: 50 mg/0.5 mL in a single-dose prefilled syringe or single-dose prefilled SmartJect® auto injector; and 100 mg/mL in a single-dose prefilled syringe or single-dose prefilled SmartJect® auto injector. The dosage forms for SIMPONI ARIA is 50 mg/4 mL (12.5 mg/mL) solution in a single-dose vial. There have been reports of severe hepatic reactions including acute liver failure in patients receiving TNF blockers such as golimumab.

Secukinumab (Cosentyx® is an IL-17A antagonist indicated for moderate to severe plaque psoriasis in adult patients who are candidates for systemic therapy or phototherapy, adults with active psoriatic arthritis (PsA), adults with active ankylosing spondylitis (AS), and adults with active non-radiographic axial spondyloarthritis (nr-axSpA) with objective signs of inflammation. For plaque psoriasis, recommended dosage is 300 mg by subcutaneous injection at weeks 0, 1, 2, 3, and 4 followed by 300 mg every 4 weeks. For some patients, a dose of 150 mg may be acceptable. For PsA patients with coexistent moderate to severe plaque psoriasis, use the dosage and administration for plaque psoriasis. For other psoriatic arthritis patients, for AK, and for nr-axSpa, administer with a loading dosage (150 mg at weeks 0, 1, 2, 3, and 4 and every 4 weeks thereafter) or without a loading dosage (150 mg every 4 weeks). If a patient continues to have active psoriatic arthritis, consider a dosage of 300 mg every 4 weeks. The dosage forms are: 150 mg/mL solution in a single-use Sensoready® pen; 150 mg/mL solution in a single-use prefilled syringe; and 150 mg, lyophilized powder in a single-use vial for reconstitution for healthcare professional use only. The sequence of secukinumab includes SEQ ID NOs: 1, 2, 5, 7, 8, 10, and 11.

Sarilumab (Kevzara®) is an IL-6 receptor antagonist indicated for adult patients with moderately to severely active rheumatoid arthritis who have had an inadequate response or intolerance to one or more disease-modifying ant rheumatic drugs (DMARDs). The recommended dosage is 200 mg once every two weeks, administered as a subcutaneous injection. KEVZARA may be used as monotherapy or in combination with methotrexate (MTX) or other conventional DMARDs. For liver enzyme abnormalities greater than 1 to 3×ULN, dosage modification of concomitant DMARDs as clinically appropriate may be considered; for greater than 3 to 5×ULN, KEVZARA treatment may be withheld until ALT less than 3 times ULN, and then can be resumed at 150 mg every two weeks and increased to 200 mg every two weeks as clinically appropriate; For greater than 5×ULN KEVZARA may be discontinued. For low ANC (500 to 1000 cells per mm³), treatment with KEVZARA may be held until ANC greater than 1000; then resumed at 150 mg every two weeks and increased to 200 mg every two weeks as clinically appropriate. KEVZARA may be discontinued for ANC less than 500. For low platelet Count (50,000 to 100,000 cells per mm³), treatment with KEVZARA may be held until platelets greater than 100,000; then resumed at 150 mg every two weeks and increased to 200 mg every two weeks as clinically appropriate. KEVZARA may be discontinued for platelet count less than 50,000. The dosage forms are 150 mg/1.14 mL or 200 mg/1.14 mL solution in a single-dose pre-filled syringe or pen Treatment with KEVZARA has been associated with a higher incidence of transaminase elevations. These elevations were transient and did not result in any clinically evident hepatic injury in clinical studies. Increased frequency and magnitude of these elevations were observed when potentially hepatotoxic drugs (e.g., MTX) were used in combination with KEVZARA. ALT/AST levels may be assessed prior to initiation of treatment and monitored 4 to 8 weeks after start of therapy and every 3 months thereafter. When clinically indicated, other liver function tests such as bilirubin may be considered. The sequence of sarilumab includes SEQ ID NOs: 1, 2, 5, 7, 8, 10, and 11.

Alemtuzumab (1BEY) (Campath®, Lemtrada®) is a CD52-directed cytolytic antibody indicated for treatment of B-cell chronic lymphocytic leukemia (B-CLL). Dosing is escalated to the recommended dose of 30 mg/day three times per week for 12 weeks. Premedication with oral antihistamine and acetaminophen is recommended prior to dosing. The dosage form for injection is 30 mg/1 mL single-dose vial. The sequence of alemtuzumab includes SEQ ID NOs: 1, 2, 5, 7, 8, 10, and 11.

Brodalumab (Siliq®) is an IL-17 receptor A (IL-17RA) antagonist indicated for moderate to severe plaque psoriasis in adult patients who are candidates for systemic therapy or phototherapy and have failed to respond or have lost response to other systemic therapies. 210 mg of Siliq is administered by subcutaneous administration at weeks 0, 1, and 2 followed by 210 mg every two weeks. The dosage form is 210 mg/1.5 mL solution for injection in a single-dose prefilled syringe. The sequence of bordalumab includes SEQ ID NOs: 1, 2, 5, 7, 8, and 10.

Guselkumab (Tremfya®) is an IL-23 blocker indicated for treatment of adult patients with moderate to severe plaque psoriasis who are candidates for systemic therapy or phototherapy, or active psoriatic arthritis. For plaque psoriasis, 100 mg is administered by subcutaneous injection at Week 0, Week 4 and every 8 weeks thereafter. For psoriatic arthritis, 100 mg administered by subcutaneous injection at Week 0, Week 4 and every 8 weeks thereafter. TRE1VIFYA can be used alone or in combination with a conventional DMARD (e.g. methotrexate). The dosage form is 100 mg/mL in a single-dose prefilled syringe or single-dose OnePress patient-controlled injector. Elevated liver enzymes have been reported. The sequence of guselkumab includes SEQ ID NOs: 10 and 11.

Tildrakizumab (Ilymya®) is an IL-23 blocker indicated for treatment of adult patients with moderate to severe plaque psoriasis who are candidates for systemic therapy or phototherapy. The recommended dose is 100 mg at weeks 0 and 4, and every twelve weeks thereafter. The dosage form is 100 mg/mL solution in a single-dose prefilled syringe.

Obinutuzumab (Gazyva®) is a CD20-directed cytolytic antibody indicated for patients with previously untreated chronic lymphocytic leukemia (CLL) (in combination with chlorambucil), patients with follicular lymphoma who relapsed after or are refractory to a rituximab-containing regimen (in combination with bendamustine followed by obinutuzumab monotherapy), and adult patients with previously untreated stage II bulky, II, or IV follicular lymphoma (in combination with chemotherapy followed by obinutuzumab monotherapy in patients achieving at least a partial remission). GAZYVA is administered only as intravenous infusion. For CLL, dosing is 100 mg on day 1 and 900 mg on day 2 of Cycle 1, 1,000 mg on day 8 and 15 of Cycle 1, and 1,000 mg on day 1 of Cycles 2-6. For follicular lymphoma, dosing is 1,000 mg on day 1, 8 and 15 of Cycle 1, 1,000 mg on day 1 of Cycles 2-6 or Cycles 2-8, and then 1,000 mg every 2 months for up to 2 years. Patients may be premedicated for infusion-related reactions and tumor lysis syndrome. The dosage form is 1,000 mg/40 mL (25 mg/mL) single dose vial. Hepatic enzyme elevations have occurred in CLL patients who received GAZYVA in clinical trials and had normal baseline hepatic enzyme levels (AST, ALT and ALP). Medications commonly used to prevent infusion-related reactions (e.g., acetaminophen) may also be implicated. Patients may be monitored for liver function tests during treatment, especially during the first cycle. Treatment interruption or discontinuation may be considered for hepatotoxicity. The sequence of obinutuzumab includes SEQ ID NOs: 1, 2, 5, 7, 8, 10, and 11.

Ofatumumab (3GIZ) (Arzerra®) is a CD20-directed cytolytic monoclonal antibody indicated for the treatment of CLL (1) in combination with chlorambucil, for the treatment of previously untreated patients with CLL for whom fludarabine-based therapy is considered inappropriate; (2) in combination with fludarabine and cyclophosphamide for the treatment of patients with relapsed CLL; (3) for extended treatment of patients who are in complete or partial response after at least two lines of therapy for recurrent or progressive CLL; and (4) for the treatment of patients with CLL refractory to fludarabine and alemtuzumab. Patients may be pre-medicated with acetaminophen, antihistamine, and corticosteroid. For previously untreated CLL in combination with chlorambucil, the recommended dosage and schedule is 300 mg on Day 1 followed by 1,000 mg on Day 8 (Cycle 1) and 1,000 mg on Day 1 of subsequent 28-day cycles for a minimum of 3 cycles until best response or a maximum of 12 cycles. For relapsed CLL in combination with fludarabine and cyclophosphamide, the recommended dosage and schedule is 300 mg on Day 1 followed by 1,000 mg on Day 8 (Cycle 1) and 1,000 mg on Day 1 of subsequent 28-day cycles for a maximum of 6 cycles. For extended treatment in CLL the recommended dosage and schedule is: 300 mg on Day 1 followed by 1,000 mg 1 week later on Day 8, followed by 1,000 mg 7 weeks later and every 8 weeks thereafter for up to a maximum of 2 years. For refractory CLL the recommended dosage and schedule is 300 mg initial dose, followed 1 week later by 2,000 mg weekly for 7 doses, followed 4 weeks later by 2,000 mg every 4 weeks for 4 dose. The dosage forms are 100 mg/5 mL single-use vial for intravenous infusion and 1,000 mg/50 mL single-use vial for intravenous infusion. The sequence of ofatumumab includes SEQ ID NOs: 1, 2, 5, 7, 8, and 10.

Ibritumomab (Zevalin®) is a CD20-directed radiotherapeutic antibody indicated for relapsed or refractory low-grade or follicular B-cell non-Hodgkin's lymphoma (NHL) and previously untreated follicular NHL who achieve a partial or complete response to first-line chemotherapy. Administration of ibritumomab is as follows. On Day 1, administer rituximab at a dose of 250 mg/m² by intravenous infusion; on Day 7, 8, or 9, administer rituximab at a dose of 250 mg/m² by intravenous infusion. If platelets are at least 150,000/mm³, within 4 hours after rituximab infusion, administer 0.4 mCi/kg (14.8 MBq per kg) Y-90 ZEVALIN by intravenous infusion. If platelets are 100,000 to 149,000/mm³ in relapsed or refractory patients, within 4 hours after rituximab infusion, administer 0.3 mCi/kg (11.1 MBq per kg) Y-90 ZEVALIN by intravenous infusion. The dosage form of ZEVALIN is 3.2 mg per 2 mL in a single dose vial. The sequence of ibritumomab includes SEQ ID NOs: 1, 5, 7, and 8.

Ocrelizumab (Ocrevus®) is a CD20-directed cytolytic antibody indicated for relapsing forms of MS, including clinically isolated syndrome, relapsing-remitting disease, and active secondary progressive disease, in adults; and primary progressive forms of MS in adults. The start dose for OCREVUS is 300 mg by intravenous infusion, followed two weeks later by a second 300 mg intravenous infusion. Subsequent doses are 600 mg by intravenous infusion, every 6 months. Patients may be premedicated with methylprednisolone (or an equivalent corticosteroid) and an antihistamine (e.g., diphenhydramine) prior to each infusion. The dosage form of OCREVUS is 300 m/10 mL (30 mg/mL) in a single dose vial.

In certain aspects of the present disclosure, there are provided methods for treating an autoimmune disease, a cancer, or other disease or disorder that may be amenable to treatment with a therapeutic agent provided herein comprising any one or more of SEQ ID NOs: 1-11. For example, the methods provided are for treating an autoimmune disease, cancer, or other disease or condition that may be amenable to treatment with one or more of the following therapeutic agents: adalimumab, belimumab, canakinumab, certolizumab, daclizumab, efalizumab, infliximab, ixekizumab, rituximab, ustekinumab, natalizumab, vedolizumab, tocilizumab, secukinumab, sarilumab, alemtuzumab, brodalumab, guselkumab, tildrakizumab, obinutuzumab, ofatumumab, ibritumomab, and ocrelizumab. In certain aspects, methods are provided for reducing the risk or severity of DILI in subjects having an autoimmune disease, cancer, or other disease or disorder that may be amenable to treatment with such therapeutic agents. The methods include reducing the dose of the therapeutic agent; prescribing a dose of the therapeutic agent that is lower than the dose on the approved label or the previously prescribed or otherwise prescribed dose; ceasing administration of the therapeutic agent; and/or not administering the therapeutic agent to the subject, which includes administering an alternative to the therapeutic agent. For example, in embodiments, the methods provided herein comprise administering the therapeutic agent to the subject in an amount of less than about 5 mg/kg, less than about 4.5 mg/kg, less than about 4 mg/kg, less than about 3.5 mg/kg, less than about 3 mg/kg, less than about 2.5 mg/kg, less than about 2 mg/kg, less than about 1.5 mg/kg, less than about 1 mg/kg, less than about 0.5 mg/kg, less than about 0.25 mg/kg, less than about 0.1 mg/kg, or less. In certain embodiments, the term “less than” a given dosing level encompasses a dose of 0 mg/kg. For example, in embodiments, a subject who is administered “less than 5 mg/kg of the therapeutic agent” is administered 0 mg/kg of the therapeutic agent. In embodiments, the methods provided herein comprise administering the therapeutic agent to the subject in an amount of less than about half of the dose that would otherwise be indicated, or less than about 10% of the dose that would otherwise be indicate, or less than about 1% of the dose that would otherwise be indicated. In certain embodiments, the term “less than” a given dosing level encompasses a dose of 0 mg or 0 mg/kg. For example, in embodiments, a subject who is administered “less than 5 mg/kg of the therapeutic agent” is administered 0 mg or 0 mg/kg of the therapeutic agent. Thus, in embodiments, administration of “less than 5 mg/kg of” an indicated therapeutic agent or “less than 50% of the dose that would otherwise be indicated” and the like includes non-administration of the therapeutic agent.

In embodiments, the subjects who are administered a reduced dose of one of the therapeutic agent comprising any one or more of SEQ ID NOs: 1-11 and/or are not administered the therapeutic agent comprising any one or more of SEQ ID NOS; 1-11, in accordance with the methods provided herein, are administered an alternative therapeutic agent to treat the disease or disorder. Thus, in embodiments, subjects are treated according to the methods provided herein with a therapeutic agent for the treatment of the disease or condition that does not include any of SEQ ID NOs: 1-11. In embodiments, the subject may be administered any drug known or suspected of treating the disease or disorder that the subject is suffering from, so long as it does not include a peptide comprising any one or more of SEQ ID NOs: 1-11. For example, in embodiments, the subject is administered a cytokine inhibitor that does not comprise any one of SEQ ID NOs: 1-11. In embodiments, the subject is administered a TNF inhibitor that does not comprise any one of SEQ ID NOs: 1-11. In embodiments, the subject is administered an immnomodulatory drug that does not comprise any of SEQ ID NOs: 1-11. In embodiments, the subject is administered a drug or therapeutic agent that does not comprise any of SEQ ID NOs: 1-11. Drugs or therapeutic agents that do not comprise any of SEQ ID NOs: 1-11 are referred to herein as “alternative therapeutic agents,” “alternative drugs,” “alternative therapies,” and the like, and include, for example a drug or therapeutic agent selected from the group consisting of azathioprine, mercaptopurine, methotrexate, mesalamine, budesonide, hyoscyamine, celecoxib, hydroxychloroquin, etanercept, prednisone, cyclosporine, meloxicam, leflunomide, sulfasalazine, abatacept, avelumab, blinatumomab, caplacizumab, golimumab, moxetumomab pasudotox, palivizumab, tildrakizumab, acitretin, apremilast, corticotropin, olsalazine, balsalazide, tacrolimus, topiramate, cholestyramine, mycophenolate mofetil, dexamethasone, hydrocortisone, cromolyn, clonidine, diclofenac, naproxen, ibuprofen, aspirin, esomeprazole, famotidine, nabumetone, etodolac, atorvastatin, anakinra, methylprednisone, triamcinolone, doxycycline, indomethacin, sulindac, tramadol, auranofin, misoprostol, ketoprofen, oxaprozin, piroxicam, salsalate, acetaminophen, hydrocodone, minocycline, flurbiprofen, cortisone, penicillamine, cyclophosphamide, diflunisal, fenoprofen, interferon gamma-1b, meclofenamate, phenytoin, tetracycline, tolmetin, hydroxyurea, betamethasone, calcipotriene, tildrakizumab, interferon-alpha, interferon-beta, glatiramier acetate, fingolimod, mitoxanthrone, azathioprine, teriflunomide, dimethylfumarate, diroximel fumarate, a tyrosine kinase inhibitor drug, a checkpoint inhibitor (e.g., cemiplimab, avelumab), a chemotherapeutic agent, or any combination thereof.

In embodiments, the therapeutic agent that is not an agent that comprises any of SEQ ID NOs: 1-11 or that is an alternative to an agent that comprises any one or more of SEQ ID NOs: 1-11 includes any salt, ester, biosimilar, generic, ether, isomer, mixture of isomers, complex, prodrug, derivative, and the like of the additional or alternative therapeutic agents provided herein. Other drugs that may treat the disease or disorder from which the subject suffers will be apparent to one of ordinary skill in the art and are encompassed by the present disclosure.

### Diseases and Disorders

In one aspect, the present disclosure provides methods for treating a subject having an autoimmune or inflammatory disease. Autoimmune or inflammatory diseases, in embodiments, are selected from the group consisting of Crohn's disease, psoriasis (including plaque psoriasis), arthritis (including rheumatoid arthritis, psoriatic arthritis, osteoarthritis, or juvenile idiopathic arthritis), multiple sclerosis (including primary progressive, secondary progressive, relapsing-remitting, and clinically isolated syndrome forms of MS), ankylosing spondylitis, spondyloarthritis, non-radiographic axial spondyloarthritis (nr-axSpA), inflammatory bowel diseases (IBD), ulcerative colitis, systemic lupus erythematosus, celiac disease, myasthenia gravis, type 1 narcolepsy, neuromyelitis optica, chorioretinopathy, pemphigus vulgaris, Behcet's disease, glomerulonephritis, type 1 diabetes mellitus, epidermolysis bullosa, Goodpasture Syndrome, uveitis, sepsis, neurodegenerative diseases, neuronal regeneration, spinal cord injury, primary and metastatic cancers, a respiratory disorder, asthma, allergic and nonallergic asthma, chronic obstructive pulmonary disease (COPD), a condition involving airway inflammation, eosinophilia, fibrosis and excess mucus production, cystic fibrosis, pulmonary fibrosis, an atopic disorder, atopic dermatitis, urticaria, eczema, allergic rhinitis, allergic enterogastritis, liver cirrhosis, liver fibrosis, and scleroderma.

In certain embodiments, the patient has an autoimmune disease. In embodiments, the patient has a disease or condition selected from Crohn's disease, pediatric Crohn's disease, ulcerative colitis, pediatric ulcerative colitis, rheumatoid arthritis, ankylosing spondylitis, psoriatic arthritis, and plaque psoriasis.

In embodiments, the patient has a disease or condition selected from the group consisting of sterile corneal melt, depression, bipolar depression (e.g., bipolar I/11 depression), major depressive disorder (MDD), refractory intestinal Behcet's disease, Takayasu's arteritis, dolichoectatic vertibrobasilar (DVB) aneurysms, refractory idiopathic scleritis, graft versus host disease, steroid refractory acute graft versus host disease, macular edema, diabetic macular edema, refractory polymyalgia rheumatic (PMR), choroidal neovascularization, Kawasaki disease, melanoma, advanced melanoma, inflammatory skin disease, sarcoidosis, and anal fistulae. In embodiments, the patient is a recipient of an organ transplant such as a kidney transplant.

In one aspect, the present disclosure provides methods for treating a subject having a cancer. For example, in embodiments, the cancer is a lymphoma, leukemia, or myeloma. In further embodiments, the lymphoma is a B-cell lymphoma or a T-cell lymphoma. In embodiments, the lymphoma is selected from the group consisting of non-Hodgkin's Lymphoma (NHL), chronic lymphocytic leukemia (CLL), follicular lymphoma, follicular NHL, Hodgkin's lymphoma, diffuse large B-cell lymphoma, Burkitt lymphoma, mantle cell lymphoma, and Waldenstrom's macroglobinemia.

### Methods

The present inventors surprisingly found that the risk of DILI upon administration of certain drugs is significantly higher in subjects having a particular HLA allele or set of alleles. Thus, the drugs identified herein are contraindicated in the patient populations provided herein. This information can be used in methods for drastically improving the treatment of patients having diseases or disorders that may be indicated for treatment with a drug that would likely cause high risk of DILI in the subject due to the subject's HLA profile. Patients can be HLA typed prior to or following initiation of treatment, and based on this information the therapeutic agents identified herein can be excluded from the patient's treatment regimen, or the patient can be administered a reduced dose or reduced dosing regimen of the therapeutic agents provided herein, or the administration of the therapeutic agent can be halted, based on the HLA typing and using the methods provided herein. Thus, in certain embodiments, the present disclosure provides new and improved methods for treating cancers, autoimmune diseases, or other diseases that may be treated with one or more of the therapeutic agents provided herein; new and improved methods for reducing the risk of DILI, and other related improved methods. In any of the embodiments provided herein including methods for reducing the risk of DILI and methods for treating a subject having a disease or condition, the methods comprise identifying the subject or a patient population as having or not having any one or more of a set of HLA alleles that comprises HLA-B*39:01, HLA-C*12:03, HLA-DRB1*03:01, HLA-DQB1*02:01, and/or HLA-DQA1*03:03.

In particular embodiments, the methods relate to a defined patient population having an HLA profile provided herein. In embodiments, the patient populations include (1) the population of patients having HLA-B*39:01 and (2) the population of patients who do not have HLA-B*39:01. In embodiments, the patient populations include (1) the population of patients having any one or more of HLA-B*39:01, HLA-C*12:03, HLA-DRB1*03:01, HLA-DQB1*02:01, and/or HLA-DQA1*03:03 and (2) the population of patients not having any one or more of HLA-B*39:01, HLA-C*12:03, HLA-DRB1*03:01, HLA-DQB1*02:01, and/or HLA-DQA1*03:03.

The therapeutic agents provided herein include any therapeutic agent comprising a peptide according to any of SEQ ID NOs: 1-11, or any combination thereof. In embodiments, the peptide is present on the surface of the therapeutic agent or otherwise located in a position such that it can interact with certain HLA alleles on cell surfaces. In embodiments, the therapeutic agents are selected from the group consisting of adalimumab, belimumab, canakinumab, certolizumab, daclizumab, efalizumab, infliximab, ixekizumab, rituximab, ustekinumab, natalizumab, vedolizumab, tocilizumab, secukinumab, sarilumab, alemtuzumab, brodalumab, guselkumab, obinutuzumab, ofatumumab, ibritumomab, and ocrelizumab.

In embodiments, the methods relating to treating a disease or disorder (e.g., an autoimmune disease, inflammatory disease, or cancer) provided herein comprise determining or obtaining the HLA profile of a subject and, if the subject has an HLA profile comprising HLA-B*39:01, then (i) not administering a therapeutic agent comprising any one or more of SEQ ID NOs: 1-11, or any combination thereof to the subject; (ii) administering less than about 5 mg/kg of the therapeutic agent to the subject or less than about 50% of the usual dose of the therapeutic agent, (iii) administering an alternative to the therapeutic agent to the subject, wherein the alternative therapeutic agent does not comprise any of SEQ ID NOs: 1-11; (iv) administering the therapeutic agent to the subject at a reduced dosing level and/or frequency relative to the level and/or frequency that the therapeutic agent would have otherwise been administered, and/or (v) closely monitoring the subject for signs or symptoms of DILI.

In embodiments, the present disclosure provides methods for reducing the risk of DILI. In further embodiments, the risk of developing DILI after administration of any therapeutic agent comprising any one or more of SEQ ID NOs: 1-11, or any combination thereof, is high in subjects having certain HLA alleles and/or combinations of alleles provided herein, e.g., in subjects having HLA-B*39:01; and the methods provided herein reduce the risk that the subject will develop DILI by providing a means for identifying the risk in a given patient or a population of patients, and reducing the risk. In embodiments, the methods for reducing the risk of DILI in a subject comprise determining or obtaining the HLA profile of the subject and, if the subject has an HLA profile comprising HLA-B*39:01, then reducing the risk of DILI by (i) not administering a therapeutic agent comprising a peptide according to any of SEQ ID NOs: 1-11, or any combination thereof, to the subject; (ii) administering less than about 5 mg/kg of such a therapeutic agent to the subject (e.g., administering less than about 4 mg/kg, less than about 3 mg/kg, less than about 2 mg/kg, less than about 1 mg/kg or 0 mg/kg of a therapeutic agent comprising a peptide according to any of SEQ ID NOs: 1-11 or any combination thereof), (iii) administering an alternative to such a therapeutic agent to the subject; (iv) administering the therapeutic agent to the subject at a reduced dosing level and/or frequency relative to the level and/or frequency that the therapeutic agent would have otherwise been administered, and/or (v) closely monitoring the subject for signs or symptoms of DILI.

In embodiments, the methods provided herein can be used to reduce the severity of DILI, and/or speed the time to recovery from DILI that has occurred in a subject following administration of a therapeutic agent comprising a peptide according to any of SEQ ID NOs: 1-11, or any combination thereof. Thus, in embodiments, the present disclosure provides methods for reducing the severity of and/or speeding recovery from DILI in a subject that has received such a therapeutic agent, comprising determining or obtaining the HLA profile of the subject and, if the subject has an HLA profile HLA-B*39:01, then reducing the risk of DILI by (i) ceasing administration of the therapeutic agent to the subject; (ii) reducing the dose of the therapeutic agent (e.g., reducing the dose to less than about 5 mg/kg, for example, administering less than about 4 mg/kg, less than about 3 mg/kg, less than about 2 mg/kg, less than about 1 mg/kg or 0 mg/kg of the therapeutic agent), (iii) administering an alternative to the therapeutic agent (e.g., an alternative therapeutic agent provided herein) to the subject, (iv) closely monitoring the subject for signs or symptoms of DILI, and/or (v) treating the subject with a steroid or other drug indicated for treatment of DILI.

Race and/or ethnicity may confer varied genetic susceptibility to DILI. Subjects having the HLA profiles provided herein may be grouped or clustered into certain races or ethnicities. In embodiments, the patient treated by the methods of the present disclosure can be characterized by two or more of the characteristics described herein. In embodiments, the patient may have an HLA profile provided herein comprising HLA-B*39:01, along with an advanced age and/or an underlying disease or infection such as infection with hepatitis B virus (HBV) and/or hepatitis C virus (HCV) and/or human immunodeficiency virus (HIV). In embodiments, the patient may be of advanced age, a high BMI, or a low BMI.

In certain embodiments, an HLA profile comprising HLA-B*39:01 confers a heightened risk of DILI upon administration of a therapeutic agent comprising a peptide according to any one or more of SEQ ID NOs: 1-11 to a patient population. In embodiments, any therapeutic agent comprising any one or more of SEQ ID NOs: 1-11 is contraindicated in the patient population having the HLA profile. In embodiments, adalimumab, belimumab, canakinumab, certolizumab, daclizumab, efalizumab, infliximab, ixekizumab, rituximab, ustekinumab, natalizumab, vedolizumab, tocilizumab, secukinumab, sarilumab, alemtuzumab, brodalumab, guselkumab, obinutuzumab, ofatumumab, ibritumomab, and ocrelizumab are contraindicated in the patient population having the HLA allele.

In embodiments, adalimumab is contraindicated in a patient population having HLA-B*39:01. In embodiments, belimumab is contraindicated in a patient population having HLA-B*39:01. In embodiments, canakinumab is contraindicated in a patient population having HLA-B*39:01. In embodiments, certolizumab is contraindicated in a patient population having HLA-B*39:01. In embodiments, daclizumab is contraindicated in a patient population having HLA-B*39:01. In embodiments, efalizumab is contraindicated in a patient population having HLA-B*39:01. In embodiments, infliximab is contraindicated in a patient population having HLA-B*39:01. In embodiments, ixekizumab is contraindicated in a patient population having HLA-B*39:01. In embodiments, rituximab is contraindicated in a patient population having HLA-B*39:01. In embodiments, ustekinumab is contraindicated in a patient population having HLA-B*39:01. In embodiments, natalizumab is contraindicated in a patient population having HLA-B*39:01. In embodiments, vedolizumab is contraindicated in a patient population having HLA-B*39:01. In embodiments, tocilizumab is contraindicated in a patient population having HLA-B*39:01. In embodiments, secukinumab is contraindicated in a patient population having HLA-B*39:01. In embodiments, sarilumab is contraindicated in a patient population having HLA-B*39:01. In embodiments, alemtuzumab is contraindicated in a patient population having HLA-B*39:01. In embodiments, brodalumab is contraindicated in a patient population having HLA-B*39:01. In embodiments, guselkumab is contraindicated in a patient population having HLA-B*39:01. In embodiments, obinutuzumab is contraindicated in a patient population having HLA-B*39:01. In embodiments, ofatumumab is contraindicated in a patient population having HLA-B*39:01. In embodiments, ibritumomab is contraindicated in a patient population having HLA-B*39:01. In embodiments ocrelizumab is contraindicated in a patient population having HLA-B*39:01.

All documents, including patents, applications, and non-patent publications cited herein are incorporated herein in their entireties for all purposes. For example, the disclosures of U.S. Pat. Nos. 10,519,227; 10,533,049; and 10,519,228; of US Application Publication No. US2019-0345240; and of International Application Publication No. PCT/US2019/031318 are incorporated herein in their entireties for all purposes.

### EXAMPLES

### Example 1. HLA Analysis of Patients with ADR Following Exposure to Infliximab

Samples from 25 Caucasians, 3 Africans, and 3 outliers subjects that had been treated with infliximab and had experienced DILI were obtained. These are referred to as “cases” here. These samples were compared to a set of 60 matched Caucasian and 6 matched African controls. The matched controls were patients who received infliximab for at least one year and did not experience DILI. The controls were matched for age, sex, ethnicity, and BMI where possible. Statistical analyses were performed to identify from these cases HLA alleles that are risk factors for the development of DILI following infliximab exposure. Significant alleles were determined using Fisher's Exact Test (FET) (p<0.05).

As a secondary comparison, the HLA profiles of the 25 Caucasian cases were compared to reference populations available at www.allelefrequencies.net. For calculating population presence rates, reference list entries were found by searching for “Caucasoid” ethnic origin subjects of gold-standard studies involving ≥100 subjects for alleles of interest on allelefrequencies.net. Sample sizes for the references represent the reported number of subjects directly typed for a given locus (i.e. USA NMDP European Caucasian reports 1,242,890 subjects typed for HLA-B and DRB1, but only 395,676 subjects typed for HLA-C; see Table 3).

In order to test for significant differences between the 31 Infliximab-DILI cases and the reference populations using Fisher's Exact Test (FET), counts of individuals carrying at least one copy of the allele in question were calculated one of two ways. If the population presence rate was available, the number of individuals was estimated by rounding the product of the population presence rate and the sample size for that HLA locus in that study. If no population presence rate was available, this number was estimated using the allele frequency rate and assuming Hardy-Weinberg equilibrium (HWE). That is, if the allele frequency is p, the number of individuals with the allele in question were calculated as [p²+2*p*(1−p)]*Nₛₐₘₚₗₑₛ, where p²+2*p*(1−p) represents the percentage of carriers if HWE is assumed, and N samples is the reference sample size for that locus.

TABLE 3
Reference List

| | Ref | Sample | |
|---|---|---|---|
| Allele | # | Size | Population Reference Study |
| B*39:01 | Ref 1 | 1,242,890 | USA NMDP European Caucasian |
| | Ref 2 | 298 | England Northwest |
| C*12:03 | Ref 1 | 395,676 | USA NMDP European Caucasian |
| | Ref 2 | 604 | UK Pop 3 |
| DRB1*03:01 | Ref 1 | 1,242,890 | USA NMDP European Caucasian |
| | Ref 2 | 141 | USA Philadelphia Caucasian |
| DQB1*02:01 | Ref 1 | 222 | USA San Antonio Caucasian |
| | Ref 2 | 141 | USA Philadelphia Caucasian |
| DQA1*3:03 | Ref 1 | 171 | USA Caucasian Bethesda |
| | Ref 2 | 155 | Netherlands pop 2 |

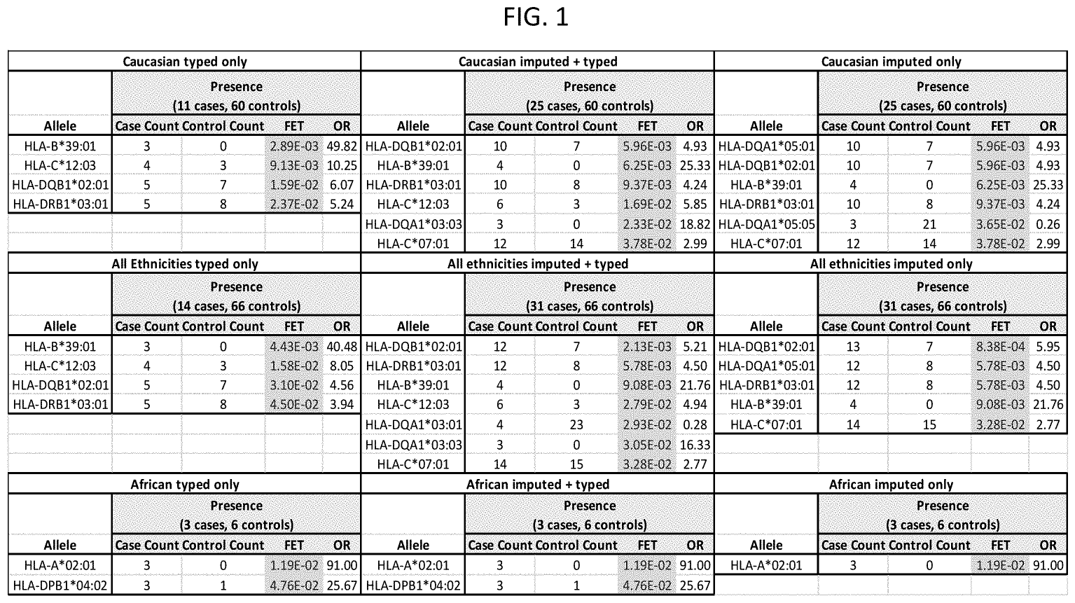

A summary of the alleles with significant results in the cases vs. controls individual data sets is provided in FIG. 1. The data sets individually analyzed were: Caucasian typed and/or Caucasian imputed; African typed and/or African imputed; and All Ethnicities typed and/or All Ethnicities imputed. “Typed only” means that only subjects with directly typed HLA alleles were considered in the indicated analysis. “Imputed only” means that only the imputed alleles (i.e., the subject's HLA profile was determined using known proxies for HLA subtypes in their genetic sequence) for each subject were used in the indicated analysis. “Imputed+Typed” means that all subjects were considered; here, directly typed HLA alleles were used if available, otherwise, imputed alleles were used.

Table 4 provides a summary of the best reported FETs and associated Odds Ratio (OR) for the indicated alleles. Of particular note, HLA-B*39:01 was present in 4/25 Caucasian cases and absent in 100% of controls, for an FET of 6.25E-03 and an OR of 25.33.

TABLE 4
Summary of best reported FET

| | Case | Case | Control | Control | | | |
|---|---|---|---|---|---|---|---|
| Variant | Present | Absent | Present | Absent | FET | OR | Dataset |
| HLA-A*02:01 | 3 | 0 | 0 | 6 | 1.19E−02 | 91.00 | African |
| | | | | | | | imputed + |
| | | | | | | | typed |
| HLA-B*39:01 | 4 | 21 | 0 | 60 | 6.25E−03 | 25.33 | Caucasian |
| | | | | | | | imputed + |
| | | | | | | | typed |
| HLA-C*12:03 | 6 | 19 | 3 | 57 | 1.69E−02 | 5.85 | Caucasian |
| | | | | | | | imputed + |
| | | | | | | | typed |
| HLA-C*07:01 | 14 | 17 | 15 | 16 | 3.28E−02 | 2.77 | All ethnicities |
| | | | | | | | imputed + |
| | | | | | | | typed |
| HLA-DQA1*03:01 | 4 | 27 | 23 | 43 | 2.93E−02 | 0.28 | All ethnicities |
| | | | | | | | imputed + |
| | | | | | | | typed |
| HLA-DQA1*03:03 | 3 | 28 | 0 | 66 | 3.05E−02 | 16.33 | All ethnicities |
| | | | | | | | imputed + |
| | | | | | | | typed |
| HLA-DQB1*02:01 | 12 | 19 | 7 | 59 | 2.13E−03 | 5.21 | All ethnicities |
| | | | | | | | imputed + |
| | | | | | | | typed |
| HLA-DPB1*04:02 | 3 | 0 | 1 | 5 | 4.76E−02 | 25.67 | African |
| | | | | | | | imputed + |
| | | | | | | | typed |
| HLA-DRB*03:01 | 12 | 19 | 8 | 58 | 5.78E−03 | 4.50 | All ethnicities |
| | | | | | | | imputed + |
| | | | | | | | typed |

Table 5A provides the overall presence rate and allele frequency of the indicated significant alleles in the cases and controls as compared to calculated and/or estimated population presence rates (Ref 1 and/or Ref 2). As above, significant alleles were determined using Fisher's Exact Test (FET) (p<0.05). The FET and associated Odds Ratio (OR) are reported in Table 5B. In some cases the population presence rate—the percentage of individuals in the population who are expected to carry at least one copy of the allele in question—is reported along with the allele frequency rate as a whole, otherwise it is given as “NR” for “not reported” (Table 5A).

TABLE 5A
Presence Rate and Allele Frequency

| | Counts | | | | | | | | | |
|---|---|---|---|---|---|---|---|---|---|---|
| | Cases | Controls | Presence Rate (%) | | | | Allele Frequency | | | |
| Allele | (25) | (60) | Cases | Controls | Ref 1 | Ref 2 | Case | Control | Ref 1 | Ref 2 |
| B*39:01 | 4 | 0 | 16% | 0.0% | NR | 1.3% | 8% | 0.0% | 1.1% | 0.7% |
| C*12:03 | 6 | 3 | 24% | 5.0% | NR | 6.5% | 12% | 2.5% | 4.9% | 3.2% |
| DRB1*03:01 | 10 | 8 | 40% | 13.3% | NR | 15.6% | 22% | 6.7% | 12.2% | 7.8% |
| DQB1*02:01 | 10 | 7 | 40% | 11.7% | NR | 15.6% | 20% | 5.8% | 11.0% | 7.8% |
| DQA1*03:03 | 3 | 0 | 12% | 0.0% | 12.9% | 13.5% | 6% | 0.0% | 7.3% | 7.0% |

TABLE 5B
Fisher’s Exact Test (FET) and Odds Ratio (OR)

| | FET vs. | | | OR vs. | | |
|---|---|---|---|---|---|---|
| Allele | Control | Ref 1 | Ref 2 | Control | Ref 1 | Ref 2 |
| B*39:01 | 0.01 | 0.00 | 0.00 | 25.33 | 8.33 | 13.72 |
| C*12:03 | 0.02 | 0.03 | 0.01 | 5.85 | 3.02 | 4.56 |
| DRB1*03:01 | 0.01 | 0.05 | 0.01 | 4.24 | 2.25 | 3.57 |
| DQB1*02:01 | 0.01 | 0.04 | 0.01 | 4.93 | 2.54 | 3.57 |
| DQA1*3:03 | 0.02 | 1.00 | 1.00 | 18.82 | 0.92 | 0.87 |

In summary, the studies showed that particular HLA alleles are associated with a higher risk of DILI following infliximab administration. In particular, HLA-B*39:01 was associated with a higher risk of DILI. The studies also showed that a set of defined HLA alleles (and/or particular two-, three-, four-, five-, six-, seven-allele, or more, combinations) are associated with higher risk of DILI following infliximab administration. The results of the study can be used to provide new means of reducing the risk of DILI in patient populations, and/or new methods for treating autoimmune disease in the patient populations

### Example 2. Peptide Interactions with HLA-B*39:01

A study was conducted to identify interactions between peptides present on the infliximab antibody and HLA-B*39:01. Peptides corresponding to heavy and light chain sequences of infliximab (chain H and L in PDB code 4G3Y) were predicted to bind class I HLA molecules based on the Stabilized matrix method (SMM) implemented in Immune Epitope Database and Analysis Resource (IEDB). Peptides predicted to bind with estimated Kd values less than 0.5 μM were considered candidate binders for: HLA-B*39:01, HLA-B*15:01, HLA-B*35:01, and HLA-B*57:01.

Refolding, characterization and purification of HLA complexes was performed as described (PMID: 12220628). Briefly, HLA-B*39:01 and β₂-microglobulin were expressed in E. coli. Inclusion bodies were isolated, denatured in 8 M Urea and mixed with synthetic peptides derived from infliximab (Genscript) at a final concentration of 0.2 mg per ml protein, 6 M Urea, 20 mM Tris pH 8.0, 1 mM GSH, 10 mM GSSH for 24 hours, then dialyzed against 10 mM Tris pH 8.0 for 3 days. Samples concentrated to reduce the volume 10 fold, then loaded on a G200 superdex column calibrated with standards (http://www.bio-rad.com/webroot/web/pdf/1sr/literature/MSLIT-102E.pdf) in 10 mM Tris, 150 mM NaCl, at 0.75 ml per minute. OD280 was recorded for chromatographic analysis.

Peptides identified as candidate peptides for binding HLA-B*39:01 with Kd<540 nM are shown in Table 6 (peptides from the infliximab light chain) and Table 7 (peptides from the infliximab heavy chain). A control peptide that is present in infliximab and that was predicted to not bind HLA-B*39:01 was included (SEQ ID NO: 12; ACEVTHQGL).

TABLE 6
Infliximab light chain peptides binding data

| | | B*39:01 | B*15:01 | B*35:01 | B*57:01 |
|---|---|---|---|---|---|
| Allele | Peptide | IC50 (nM) | IC50 (nM) | IC50 (nM) | IC50 (nM) |
| HLA-B*39:01 | THQGLSSPV | 19.97 | 31768.74 | 57488.37 | 595415.31 |
| | (SEQ ID NO: 1) | | | | |
| HLA-B*39:01 | YSLSSTLTL | 152.55 | 916.22 | 262.77 | 2587.14 |
| | (SEQ ID NO: 5) | | | | |
| HLA-B*39:01 | TQSPAILSV | 283.40 | 587.49 | 34880.28 | 64986.03 |
| | (SEQ ID NO: 6) | | | | |
| HLA-B*39:01 | STYSLSSTL | 350.27 | 532.11 | 3270.24 | 2956.79 |
| | (SEQ ID NO: 7) | | | | |
| HLA-B*39:01 | VQWKVDNAL | 364.25 | 812.83 | 3886.69 | 49982.73 |
| | (SEQ ID NO: 8) | | | | |
| HLA-B*39:01 | CQQSHSWPF | 540.01 | 21.53 | 351.22 | 34980.02 |
| | (SEQ ID NO: 9) | | | | |

TABLE 7
Infliximab heavy chain peptides binding data

| | | B*039:01 | B*15:01 | B*35:01 | B*57:01 |
|---|---|---|---|---|---|
| Allele | Peptide | IC50 (nM) | IC50 (nM) | IC50 (nM) | IC50 (nM) |
| HLA-B*39:01 | LQSSGLYSL | 83.06 | 165.20 | 3931.70 | 50097.96 |
| | (SEQ ID NO: | | | | |
| | 10) | | | | |
| HLA-B*39:01 | TKGPSVFPL | 120.06 | 25527.01 | 19124.06 | 61350.77 |
| | (SEQ ID NO: | | | | |
| | 2) | | | | |
| HLA-B*39:01 | STSGGTAAL | 278.87 | 1002.31 | 2035.07 | 11010.83 |
| | (SEQ ID NO: | | | | |
| | 11) | | | | |

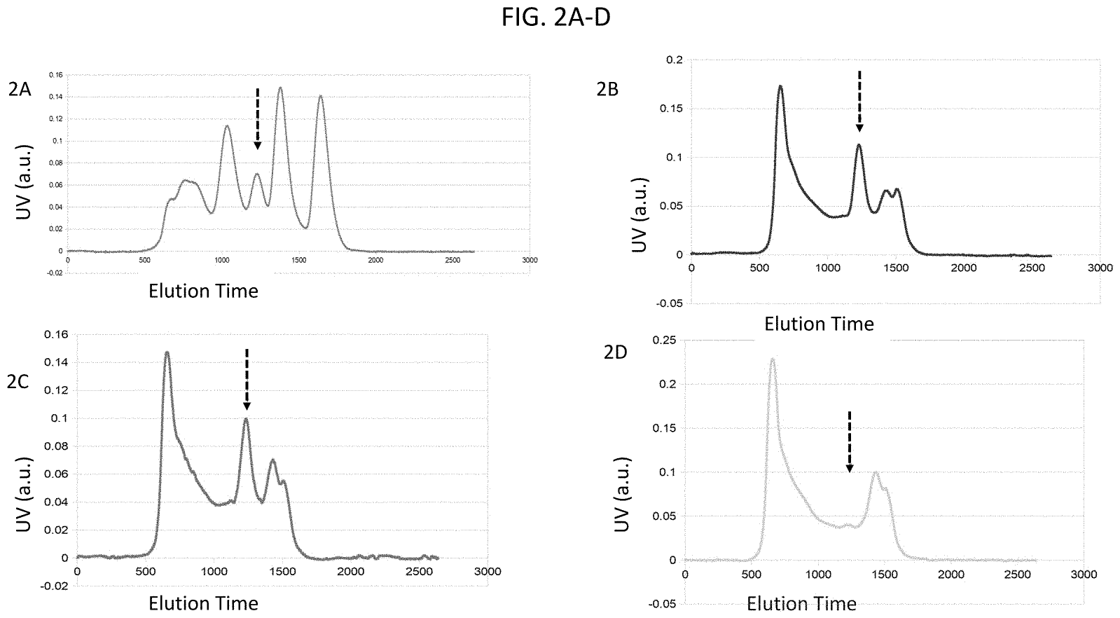

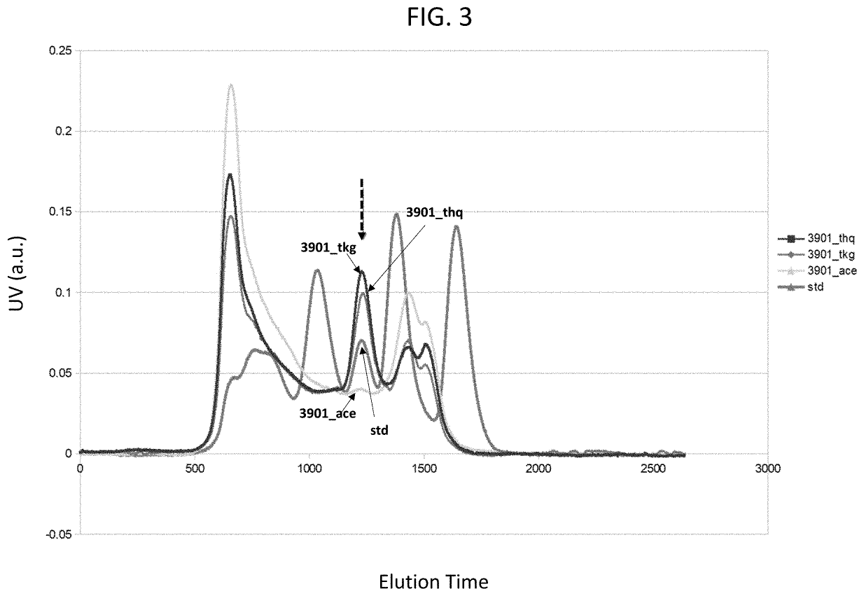

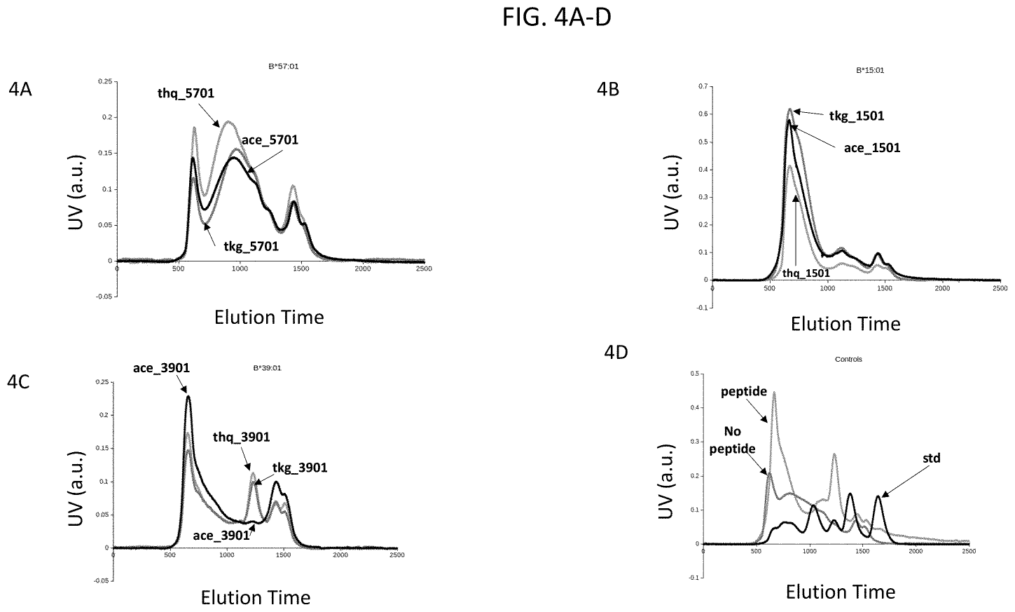

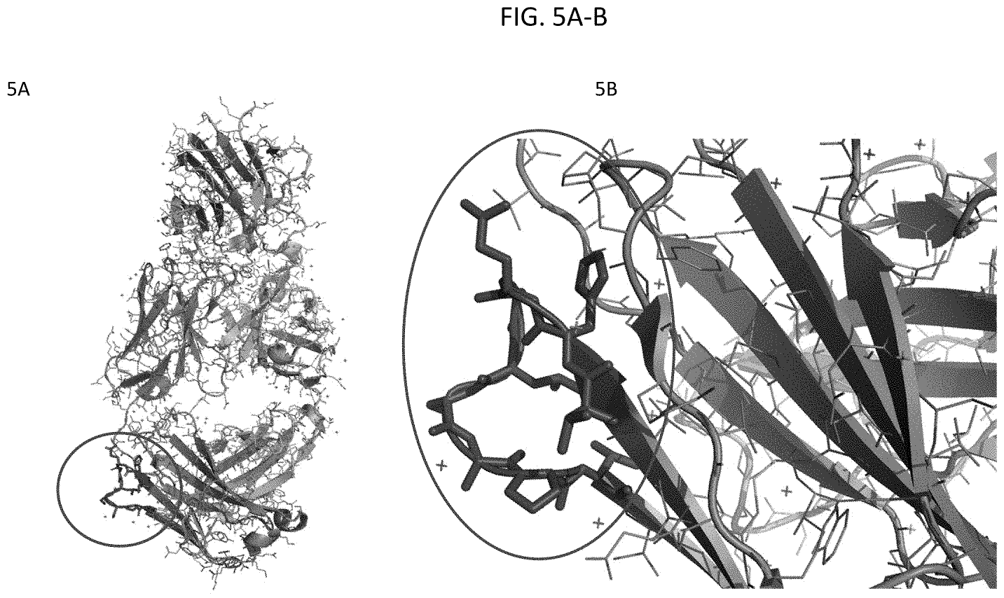

Results of the study are provided in FIGS. 2-4 and in Tables 6 and 7. In Tables 6 and 7, the IC50 (nM) for each peptide binding to HLA-B*39:01, HLA-B*05:01, HLA-B*35:01, or HLA-B*57:01 are shown in nM. FIG. 2 shows the elution standards (FIG. 2A), binding of SEQ ID NO: 1 to HLA-B*39:01 (FIG. 2B), binding of SEQ ID NO: 2 to HLA-B*39:01 (FIG. 2C), and lack of binding of control peptide SEQ ID NO: 12 to HLA-B*39:01 (FIG. 2D). In each of FIG. 2A-2D, the dotted line arrow points to the peak (or lack of a peak, for FIG. 2D) indicating peptide binding and proper refolding. FIG. 3 shows the results of FIGS. 2A-2D in one graph for comparison purposes. The dotted line arrow points to the peak (or lack of a peak) indicating peptide binding and proper refolding. FIG. 4A-4C shows the binding of the control peptide (SEQ ID NO: 12; ace in the figure), SEQ ID NO: 1 (thq in the figure), or SEQ ID NO: 2 (tkg in the figure) to HLA-B*57:01 (FIG. 4A), HLA-B*15:01 (FIG. 4B), or HLA-B*039:01 (FIG. 4C). FIG. 4D shows the elution standards, no peptide, or peptide binding to HLA-B*15:01 to show that the refolding experiment was working properly. FIGS. 5A and 5B show an exemplary crystal structure of infliximab. The circled portion shows the location of SEQ ID NO: 1.

Taken together, the results of the studies showed that peptides according to SEQ ID NOs: 1, 2, 5, 6, 7, 8, 9, 10, and 11 bind to HLA-B*39:01 at significant levels, but not to other HLAs; and that a control peptide does not bind to HLA-B*39:01. Accordingly, the study shows that therapeutic agents comprising any of the peptides according to SEQ ID NOs: 1, 2, and 5-11 interact with HLA-B*39:01 and may trigger DILI upon administration to a subject having HLA-B*39:01.

1. A method for reducing the risk of drug-induced liver injury (DILI) in a subject, comprising first determining that the subject has an HLA profile that does not include an HLA-B*39:01 allele, then administering to the subject a therapeutically effective amount of a therapeutic agent, wherein the therapeutic agent is a protein that comprises an amino acid sequence according to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or any combination thereof.

2. A method of reducing the risk of DILI in a subject, comprising:
(i) identifying the subject as not having a genetic variation comprising the HLA-B*39:01 allele; and
(ii) administering a therapeutically effective amount of a therapeutic agent to the subject, wherein the therapeutic agent is a protein that comprises an amino acid sequence according to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or any combination thereof;
whereby the subject has a decreased risk of DILI compared to an otherwise identical subject having the genetic variation.

3. A method for reducing the risk of DILI in a subject comprising treating the subject with a therapeutic agent, wherein the therapeutic agent is a protein that comprises an amino acid sequence according to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or any combination thereof, the method comprising the steps of:
(i) obtaining or having obtained the HLA profile of the subject; and
(ii) if the HLA profile of the subject does not comprise HLA-B*39:01, then administering the therapeutic agent to the subject, and if the HLA profile of the subject comprises HLA-B*39:01, then administering to the subject an alternative therapeutic that does not comprise an amino acid sequence according to any of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11.

4. The method of any one of claims 1-3, wherein the therapeutic agent is an antibody or antibody fragment.

5. The method of any one of claims 1-3, wherein the therapeutic agent is selected from the group consisting of abciximab, adalimumab, alemtuzumab, alirocumab, atezolizumab, atoltivimab, maftivimab, odesivimab, basiliximab, belantamab mafodotin, belimumab, benralizumab, bevacizumab, bezlotoxumab, brentuximab vedotin, brodalumab, brolucizumab, burosumab, canakinumab, capromab pendetide, cemiplimab, certolizumab pegol, cetuximab, crizanlizumab, daclizumab, daratumumab, denosumab, dinutuximab, dupilumab, durvalumab, eculizumab, efalizumab, elotuzumab, emapalumab, emicizumab, enfortumab vedotin, erenumab, evolocumab, fanolesomab, fremanezumab, galcanezumab, gemtuzumab ozogamicin, guselkumab, ibalizumab, ibritumomab tiuxetan, idarucizumab, inebilizumab, infliximab, inotuzumab ozogamicin, ipilimumab, isatuximab, ixekizumab, lanadelumab, mepolizumab, mogamulizumab, natalizumab, naxitamab, necitumumab, nivolumab, nofetumomab, obiltoxaximab, obinutuzumab, ocrelizumab, ofatumumab, olaratumab, omalizumab, panitumumab, pembrolizumab, pertuzumab, polatuzumab vedotin, ramucirumab, ranibizumab, ravulizumab, raxibacumab, reslizumab, risankizumab, rituximab, romosozumab, sacituzumab govitecan, sarilumab, satralizumab, secukinumab, siltuximab, tafasitamab, teprotumumab, tocilizumab, tositumomab, trastuzumab, ustekinumab, vedolizumab, tisotumab vedotin, epcoritamab, amivantamab, teclistamab, camidanlumab tesirine, talquetamab, actimab-a, lintuzumab.

6. A method for reducing the risk of DILI in a subject, wherein the subject has an HLA profile comprising HLA-B*39:01, wherein the method comprises
(i) determining that the subject has an HLA profile comprising HLA-B*39:01 by obtaining or having obtained the HLA profile of the subject, and
(ii) administering to the subject a therapeutically effective amount of a therapeutic agent for the treatment of the autoimmune disease, wherein the therapeutic agent is not a protein that comprises an amino acid sequence according to any of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11.

7. A method of reducing the risk of DILI in a population of patients having a disease or condition, the method comprising
(i) providing a first population of patients having the disease or disorder;
(ii) excluding only patients having an HLA profile comprising HLA-B*39:01 from the first population of patients;
(iii) treating the population of patients not excluded from the first population with a therapeutically effective amount of a therapeutic agent, wherein the therapeutic agent is a protein comprising an amino acid sequence according to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or any combination thereof; and
(iv) treating the excluded patients with a therapeutically effective amount of an alternative therapeutic agent, wherein the alternative therapeutic agent is not a protein comprising an amino acid sequence according to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, or SEQ ID NO: 11.

8. A method of reducing the risk of DILI in a subject, comprising:
(i) identifying the subject as having a genetic variation comprising the HLA-B*39:01 allele; and
(ii) administering a therapeutically effective amount of a therapeutic agent to the subject, wherein the therapeutic agent is not a protein that comprises an amino acid sequence according to any of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, or SEQ ID NO: 11.

9. A method for treating a disease or condition in a subject, comprising first determining that the subject has an HLA profile that includes an HLA-B*39:01 allele, then administering to the subject a therapeutically effective amount of a therapeutic agent, wherein the therapeutic agent is not a protein that comprises an amino acid sequence according to any of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, or SEQ ID NO: 11.

10. The method of any one of claims 6-9, wherein the therapeutic agent that does not comprise an amino acid sequence according to any of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, or SEQ ID NO: 11 is selected from the group consisting of azathioprine, mercaptopurine, methotrexate, mesalamine, budesonide, hyoscyamine, celecoxib, hydroxychloroquin, etanercept, prednisone, cyclosporine, meloxicam, leflunomide, sulfasalazine, abatacept, avelumab, blinatumomab, caplacizumab, golimumab, moxetumomab pasudotox, palivizumab, tildrakizumab, acitretin, apremilast, corticotropin, interferon-alpha, interferon-beta, glatiramier acetate, fingolimod, mitoxanthrone, azathioprine, teriflunomide, and dimethylfumarate.

11. The method of any one of claims 6-10, wherein the disease is multiple sclerosis, and wherein the therapeutic agent that does not comprise an amino acid sequence according to any of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, or SEQ ID NO: 11 is selected from the group consisting of interferon-alpha, interferon-beta, glatiramier acetate, fingolimod, mitoxanthrone, azathioprine, teriflunomide, dimethylfumarate.

12. The method of any one of claims 6-10, wherein the disease is rheumatoid arthritis, ankylosing spondylitis, or psoriatic arthritis, and wherein the therapeutic agent that does not comprise an amino acid sequence according to any of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, or SEQ ID NO: 11 is selected from the group consisting of methotrexate, abatacept, etanercept, and golimumab.

13. The method of any one of claims 6-10, wherein the disease is Crohn's disease or ulcerative colitis, and wherein the therapeutic agent that does not comprise an amino acid sequence according to any of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, or SEQ ID NO: 11 is selected from the group consisting of methotrexate, azathioprine, cyclosporine, and mercaptopurin.

14. The method of any one of claims 1-9, wherein the disease or condition is an autoimmune disease, an inflammatory disease, or a cancer.

15. The method of claim 14, wherein the autoimmune disease or inflammatory disease is selected from the group consisting of Crohn's Disease, Ulcerative Colitis; rheumatoid arthritis, polyarticular or systemic juvenile idiopathic arthritis, ankylosing spondylitis; psoriatic arthritis, plaque psoriasis, psoriasis vulgaris, pustular psoriasis, erythrodermic psoriasis uveoretinitis, panuveitis, Behcet's disease; pustular psoriasis, erythrodermic psoriasis, hidradenitis suppartiva; panuveitis, granulomatosis with polyangitis (Wegner's granulomatosis), systemic lupus erythematosus, and multiple sclerosis.

16. The method of claim 14, wherein the cancer is a lymphoma.

17. The method of claim 14, wherein the cancer is selected from non-Hodgkin's Lymphoma (NHL), chronic lymphocytic leukemia (CLL), follicular lymphoma, and diffuse large B-cell lymphoma.

18. The method of any one of claims 1-17, comprising performing a genetic assay to determine the presence or absence of the HLA-B*39:01 allele.

19. The method of claim 18, wherein the genetic assay comprises one or more of a polymerase chain reaction (PCR)-based approach, a direct sequencing approach, a next generation (NGS) approach and/or a direct HLA typing test.

20. The method of claim 18, wherein the genetic assay comprises obtaining a PCR-amplified genomic DNA sample of the biological sample from the subject, contacting under hybridizing conditions the genomic DNA with an oligonucleotide that specifically hybridizes to HLA-B*039:01, and detecting the presence of HLA-B*039:01 in the sample.
